(19) United States
(12) Patent Application Publication
(10) Pub. No.: US 2026/0033750 A1
(43) Pub. Date: Feb. 5, 2026

(54) DETERMINATION OF A USER’S GLYCATED HEMOGLOBIN LEVEL USING PULSE SPECTROSCOPY

(71) Applicant: Garmin International, Inc., Olathe, KS (US)

(72) Inventors: Paul R. MacDonald, Cochrane (CA); Christopher J. Kulach, Cochrane (CA)

(21) Appl. No.: 18/731,841

(22) Filed: Jun. 3, 2024

Related U.S. Application Data
(60) Provisional application No. 63/505,554, filed on Jun. 1, 2023.

(51) Int. Cl. A61B 5/145; A61B 5/00; A61B 5/0205; A61B 5/024; A61B 5/1455

(52) U.S. Cl. CPC A61B 5/14532; A61B 5/0205; A61B 5/14551; A61B 5/681; A61B 5/02433

(57) ABSTRACT

An electronic device comprises a housing configured to contact a user's skin, an optical transmitter array, an optical receiver, a memory element and a processing element. The optical transmitter array outputs a plurality of optical signals that pass through the user's skin, each of the plurality of optical signals having one of three wavelengths. The optical receiver receives the optical signals and generates corresponding photoplethysmogram (PPG) signals. The memory element stores absorption constants for deoxyhemoglobin blood content, oxyhemoglobin blood content and glycated hemoglobin blood content, each absorption constant associated with one of the first wavelength, the second wavelength or the third wavelength. The processing element determines a glycated hemoglobin blood level for the user based on a ratio of a determined glycated hemoglobin blood content to a sum of determined deoxyhemoglobin blood content, a determined oxyhemoglobin blood content and a determined glycated hemoglobin blood content.

### RELATED APPLICATIONS

This patent application is a non-provisional utility patent application which claims priority benefit, with regard to all common subject matter, under 35 U.S.C. § 119 (c) of earlier-filed U.S. provisional application 63/505,554, filed Jun. 1, 2023, and entitled “WRIST-BASED DETERMINATION OF A USER'S GLYCATED HEMOGLOBIN USING PULSE SPECTROSCOPY.” The Provisional Application is hereby incorporated by reference in its entirety.

### BACKGROUND

An electronic wearable device may provide optical cardiac monitoring of a user of the device. The user may wear the electronic device such that a housing of the electronic device is located in contact with the skin of the user-typically being worn on the user's wrist. The cardiac monitoring may include physiological metrics and information such as a user's heart rate and pulse oximetry.

Pulse spectroscopy is the observation and analysis of optical signals that are directed at human skin in order to determine blood-related and cardiac physiological metrics and information, such as a user's glycated hemoglobin (HbA1c), blood oxygen saturation (SpO2), and heart rate. Red blood cells contain oxygenated hemoglobin (oxyhemoglobin, O2Hb) and deoxygenated hemoglobin (deoxyhemoglobin, HHb).

A user's glycated hemoglobin level, also known as HbA1c and A1c, is a volume percentage (vol %) of hemoglobin in an individual's blood that is glycated (i.e., hemoglobin to which glucose is bound or over which glucose is coated). Hemoglobin is a protein in red blood cells that transports oxygen from the lungs to all parts of the body. Once glucose (sugar) is bound or attached to hemoglobin (resulting in glycated hemoglobin), the glucose stays attached to the hemoglobin for the life of that red blood cell, which is typically four months. Accordingly, an individual's glycated hemoglobin level provides a long-term indication of the average blood glucose level for the individual (over the past three months) and is typically not impacted substantially by a single meal or activity. As the level of glycated hemoglobin (HbA1c) changes slowly over time, an elevated level of glycated hemoglobin in a person's blood may be an indication of prediabetes or diabetes. In contrast, a user's blood oxygen saturation increases as the concentration of oxygenated hemoglobin increases. Unlike a user's glycated hemoglobin level (HbA1c), his or her blood oxygen saturation levels can change rapidly during activities (almost instantaneously compared to the rate at which glycated hemoglobin levels (HbA1c) typically change).

A widely-accepted healthy (or normal) level of glycated hemoglobin (HbA1c) is typically below 5%. Glycated hemoglobin (HbA1c) levels above 7% or 10%, depending on each individual's health considerations and circumstances, may present a concern for an individual developing diabetes, which can also affect the kidneys and other organs of those individuals over the long-term. For an individual who has prediabetes or diabetes, determining and monitoring glycated hemoglobin levels (HbA1c) can help that individual with the management of glucose and insulin (as it is common for diabetics to maintain a glycated hemoglobin level under 7%). Lowering (and maintaining) the glycated hemoglobin level (HbA1c) below 5% is believed to improve and maintain an individual's overall health as elevated levels may be associated with poor control of blood sugar and higher risk of diabetes.

### SUMMARY

Embodiments of the present technology relate to an electronic device that determines a user's glycated hemoglobin (HbA1c) level. An embodiment of the electronic device broadly comprises a housing, an optical transmitter array, an optical receiver, a memory element and a processing element. The housing includes a bottom wall configured to contact a user's wrist. The optical transmitter array is positioned at a first location on the bottom wall and operable to output a plurality of optical signals that pass through a user's skin, the optical transmitter array including a first optical transmitter configured to transmit a first optical signal having a first wavelength, a second optical transmitter configured to transmit a second optical signal having a second wavelength and a third optical transmitter configured to transmit a third optical signal having a third wavelength. The optical receiver is positioned at a second location on the bottom wall and operable to receive the optical signals modulated by the skin of the user and generate first, second and third photoplethysmogram (PPG) signals resulting from the received first, the second and the third optical signals, respectively, the first optical signal, the second optical signal and the third optical signal each traveling along substantially the same path and each optical signal containing a cardiac component of the user. The memory element is configured to store absorption constants for deoxyhemoglobin blood content, oxyhemoglobin blood content and glycated hemoglobin blood content, each absorption constant associated with one of the first wavelength, the second wavelength or the third wavelength. The processing element is in electronic communication with the optical transmitter array and the optical receiver. The processing element is configured to receive the first, the second and the third PPG signals from the optical receiver, determine a first AC-to-DC ratio for the first PPG signal, determine a second AC-to-DC ratio for the second PPG signal, determine a third AC-to-DC ratio for the third PPG signal. The processing element is further configured to determine, based on the three determined AC-to-DC ratios, the deoxyhemoglobin blood content, the oxyhemoglobin blood content and the glycated hemoglobin blood content and determine a glycated hemoglobin blood level (HbA1c) for the user based on a ratio of the determined glycated hemoglobin blood content to a sum of the determined deoxyhemoglobin blood content, the determined oxyhemoglobin blood content and the determined glycated hemoglobin blood content.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present technology will be apparent from the following detailed description of the embodiments and the accompanying drawing figures.

### BRIEF DESCRIPTION OF THE DRAWINGS

Embodiments of the current technology are described in detail below with reference to the attached drawing figures, which are referenced in the detailed description. The use of the same reference numbers in different instances in the description and the figures may indicate similar or identical items.

FIG. 1 is a top view of a wrist-worn electronic device, constructed in accordance with various embodiments of the present technology, worn on a user's wrist.

FIG. 2 is a plot of a photoplethysmogram (PPG) signal waveform that may be generated by the electronic device over a period of time.

FIG. 3A is a schematic side sectional view of the electronic device and a user's wrist depicting transmission of an optical signal through the skin and tissue of the user.

FIG. 3B illustrates a side view of a wrist-worn electronic device.

FIG. 3C is a bottom view of the wrist-worn electronic device, illustrating a plurality of optical transmitter arrays and a plurality of optical receivers.

FIG. 4 is a plot of a cardiac component of the PPG signal resulting from the PPG signal being filtered.

FIGS. 5A-5B are plots of an absorption coefficient, or level, of the optical signal versus a wavelength of the optical signal, wherein the optical signal may be absorbed by various components of blood, such as oxygenated blood and deoxygenated blood.

FIG. 5C is a plot of an absorption coefficient, or level, of the optical signal versus a wavelength of the optical signal, wherein the optical signal may be absorbed by various components of blood, such as oxygenated blood and deoxygenated blood.

FIG. 5D is a plot of an absorption coefficient, or level, of the optical signal versus a wavelength of the optical signal, wherein the optical signal may be absorbed by various components of blood, such as oxygenated blood and deoxygenated blood.

FIG. 6 is a schematic view of a first embodiment of a plurality of optical transmitter arrays and optical receivers illustrating pathways of the optical signal transmitted by each of the optical transmitters and received by one of a plurality of optical receivers of a wrist-worn electronic device.

FIG. 7 is a block hardware diagram of various components of the wrist-worn electronic device.

FIGS. 8A and 8B are a flow chart of at least a portion of the steps of a process for determining a glycated hemoglobin blood level (HbA1c), a fractional blood oxygen saturation level (Fractional SpO2) and/or a functional blood oxygen saturation level (Functional SpO2) in accordance with embodiments of the technology.

The drawing figures do not limit the current invention to the specific embodiments disclosed and described herein. The drawings are not necessarily to scale, emphasis instead being placed upon clearly illustrating the principles of the invention.

### DETAILED DESCRIPTION OF THE INVENTION

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the current invention. The following detailed description is, therefore, not to be taken in a limiting sense. The scope of the current technology is defined only by the appended claims, along with the full scope of equivalents to which such claims are entitled.

Relational and/or directional terms, such as “above”, “below”, “up”, “upper”, “upward”, “down”, “downward”, “lower”, “top”, “bottom”, “outer”, “inner”, etc., along with orientation terms, such as “horizontal” and “vertical”, may be used throughout this description. These terms retain their commonly accepted definitions and are used with reference to embodiments of the technology and the positions, directions, and orientations thereof shown in the accompanying figures. Embodiments of the technology may be positioned and oriented in other ways or move in other directions. Therefore, the terms do not limit the scope of the current technology.

It is to be understood that the electronic device described herein may provide optical cardiac monitoring of a user of the electronic device once positioned against any portion of the user's body. Accordingly, the user (wearer) may be any individual who wears the electronic device such that a housing is coupled with a band that enables the electronic device to be located against or proximate to skin of the individual's wrist, upper arm, abdomen, leg, finger (e.g., over a fingertip, around a lower base of a finger similar to a ring, etc.), forehead, earlobe, or any other body part through which optical signals may pass from an optical transmitter to an optical receiver. For example, the electronic device may be a fitness watch, a fitness armband, a body-worn smart phone, a body-worn navigation device, or other wearable multi-function electronic device that include a housing and a band, wrist band, strap, clip, ring, or other attachment mechanism to secure the electronic device to an extremity (e.g., wrist, finger, forearm, upper arm, leg, etc.) of the user.

Cardiac monitoring implemented by the electronic device may include physiological metrics and information such as a user's glycated hemoglobin level (HbA1c), fractional blood oxygen saturation level (Functional SpO2) or functional blood oxygen saturation level (Functional SpO2), or heart rate. The electronic device may utilize a photoplethysmogram (PPG) signal to determine the cardiac monitoring information. The PPG signal is typically output by a photodiode and is commonly utilized to identify changes in the volume of blood in the skin proximate to the photodiode and is collected over a period of time encompassing a plurality of heart beats. The electronic device may include optical devices, such as an optical transmitter, which emits an optical signal (light) into the user's skin, and an optical receiver, which receives reflections of the optical signal (light) from the skin and generates a PPG signal corresponding to the intensity of the received light. Typically, the electronic device includes a housing and straps enabling it to be worn on the user's wrist, arm, leg, or torso, and the optical devices are positioned on the back, or bottom wall, of the housing to orient the optical devices to output and receive light from the user's skin when the device is worn.

Many conventional body-worn devices include a housing having a bottom surface containing optical transmitters (e.g., LEDs) that emit (output) light into a user's extremity (e.g., wrist, upper arm, leg, etc.) and optical receivers (photodiodes) that receive reflections of the emitted light. The devices typically include one or more processing elements that generate PPG signals associated with the intensity of reflected light and use the generated PPG signals to determine cardiac information about the user, including physiological metrics, such as a user's blood oxygen saturation level or heart rate. A transmitter array may form a plurality of optical transmitters, some of which emit light at a different wavelength than the light emitted by other optical transmitters. It is common for conventional body-worn electronic devices to incorporate a plurality of transmitter arrays and a plurality of optical receivers at different locations, which enables optical signals to pass through many paths and use different wavelengths of light. The techniques used to control the output of optical signals and positioning of optical components may impact the quality of reflections received by the optical sensor(s) and the corresponding PPG signal, which likely impacts the accuracy of measurements and determinations of cardiac information about the user. Many optical layouts and topologies are known.

Conventional techniques for determining an individual's glycated hemoglobin level (HbA1c) typically involve use of specialized equipment or devices to perform measurements on blood that has been collected from the individual either using a finger stick or collected in a vial (commonly referred to as an “A1c test”). For example, a test may be performed at home or in a laboratory on blood that has been collected using the finger stick, a needle or other process. Accordingly, it would be of great benefit to users of an electronic device having optical transmitters that emit light having certain wavelengths into a user's extremity and optical receivers that receive reflections of the emitted light from the extremity to determine a glycated hemoglobin level (HbA1c) or receive general feedback on the overall health of the user on the determined glycated hemoglobin level (HbA1c).

Spectroscopic methods and techniques may be utilized to analyze characteristics a variety of materials and substances based on an interaction of electromagnetic radiation (light) with the materials and substances. For example, spectroscopy may be applied to the absorption, emission, and scattering of visible, infrared (IR), ultraviolet (UV), X-ray, microwave, and radio wave electromagnetic radiation with various materials and substances. Optical spectroscopy techniques may be applied using optical materials to disperse and/or focus visible, IR and UV light with materials and substances, such as the skin or tissue of a user. The electromagnetic radiation (light) oscillates as it travels and a wavelength of the oscillating electromagnetic radiation may be within a band associated with one of the visible, infrared (IR), and ultraviolet (UV) spectrum. The properties of certain materials and substances may influence the electromagnetic radiation as it passes through those materials and substances. As a result, the impact of particular materials and substances on electromagnetic radiation of certain wavelengths is generally known or otherwise measurable.

Conventional spectroscopy methods and techniques may include correlated spectroscopy, which includes analysis by comparing correlated signals, as well as augmented spectroscopy, which includes applying information determined from a reference signal to analyze another signal. Optical spectroscopy techniques applied to determine pulsatile blood-related and cardiac physiological metrics and information of a user may be referred to as optical “pulse” spectroscopy.

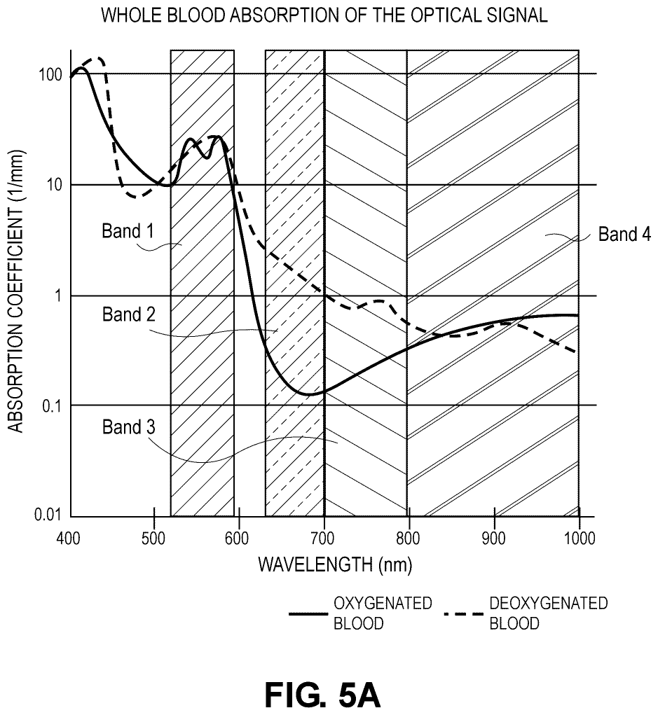

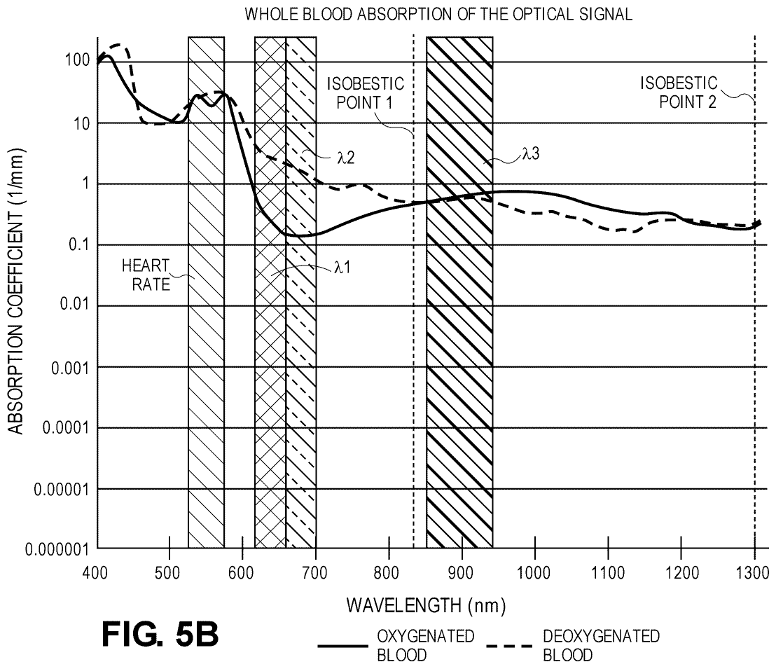

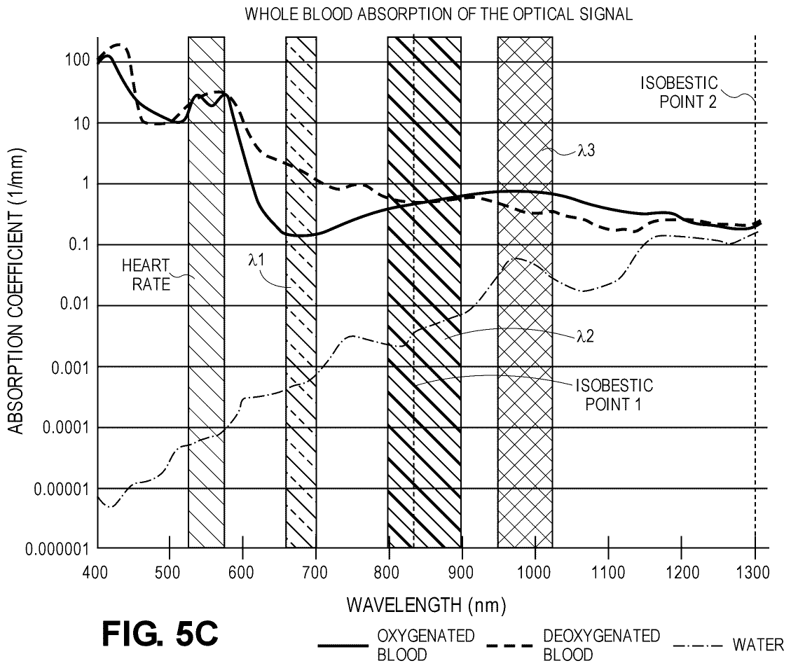

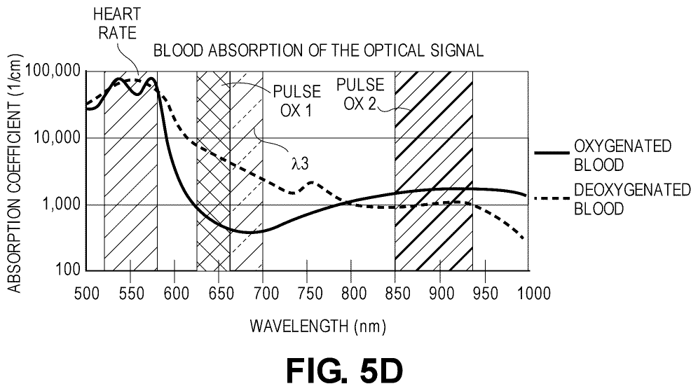

As shown in FIGS. 5A-5D, plots of an absorption level of an optical signal versus a wavelength of the optical signal are provided for oxygenated blood, deoxygenated blood, and water. Optical “pulse” spectroscopy techniques applied to determine blood-related and cardiac physiological metrics and information, such as a user's heart rate, a blood oxygen saturation level, a hematocrit level, and the like, may utilize one or more optical signals having wavelengths that are selected based on a relationship between the absorption level of an optical signal in oxygenated blood, deoxygenated blood, water, or any combination thereof. For example, some conventional optical spectroscopy techniques utilize the absorption level for oxygenated blood and deoxygenated blood to determine blood-related and cardiac physiological metrics and information by emitting one or more optical signals having a wavelength corresponding to points within the plot of FIGS. 5B-5D at which the absorption level of the oxygenated and deoxygenated blood is high (e.g., the band labeled “heart rate”), the least variance or separation (e.g., an intersection point at which the absorption level of the oxygenated and deoxygenated blood is substantially equal, such as the points labeled “isobestic point 1” and “isobestic point 2”), or any other wavelength at which the absorption level of the oxygenated and deoxygenated blood is measurable.

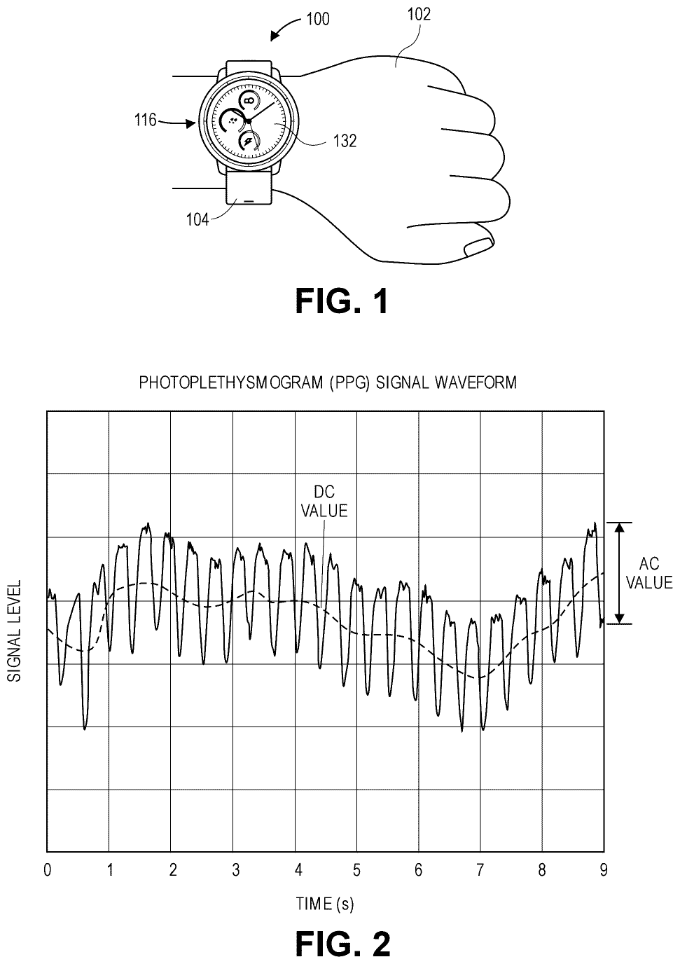

Embodiments of the present technology relate to an electronic device that may be worn on a user's body, such as the wrist-worn electronic device shown in FIG. 1, and noninvasively determine a glycated hemoglobin level (HbA1c) for the user and provide general feedback on a user interface, such as a display. As shown in FIGS. 5A-5D, the absorption of blood having red blood cell proteins having oxygenated blood (O2Hb) and deoxygenated blood (HHb) is different for optical signals (light) having different wavelengths of light. Although example wavelength bands are shown in FIGS. 5A-5D, it is to be understood that the boundaries of each band could change and implementations in accordance with those variations would be embodiments hereof.

In the example shown in FIG. 5A, a range of wavelengths (labeled “Band 1”) may exist between roughly 500 nm and 600 nm. Although optical signals (light) in Band I correspond to a high absorption level and is visible to the human eye as the color green, the optical signals do not penetrate into some of the lower layers of a user's skin (as light in Band 1 typically remains in the upper layers of the skin). In contrast, optical signals having a wavelength above 600 nm penetrate deeper (lower) layers of the user's skin tissue. In the example, the emission (output) by optical transmitters of three or more optical signals (light) having a wavelength in Band 2, Band 3 and/or Band 4 into a user's extremity may enable a processing element to determine a glycated hemoglobin level (HbA1c) for the user based on three corresponding PPG signals generated by one or more optical receivers. The optical signals output by the optical transmitters may be visible to the human eye as the color red or invisible to the human eye in the infrared band (or lower microwave band). In embodiments, three optical transmitters may output a first optical signal having a first wavelength in Band 2, a second optical signal having a second wavelength in Band 2 and third optical signal having a third wavelength in Band 4. In other embodiments, three optical transmitters may output a first optical signal having a first wavelength in Band 2, a second optical signal having a second wavelength in Band 3 and third optical signal having a third wavelength in Band 4. Additional embodiments are discussed below.

The electronic device includes at least one optical transmitter array and at least one optical receiver. Each optical transmitter array is configured to output optical signals having a plurality of wavelengths. The optical signals pass through the user's skin and are received once they exit the user's skin by a plurality of optical receivers. Each optical receiver is configured to generate an electronic PPG signal for each optical signal received. The PPG signals are communicated to the processing element which processes the PPG signals to determine the user's heart rate, hemoglobin blood levels (Hb) and other physiological information.

In embodiments, the processing element of the electronic device may determine blood content of deoxyhemoglobin (HHb) (referred to as V_{HHb}), blood content of oxyhemoglobin (referred to as V_{O2Hb}) and blood content of glycated hemoglobin (HbA1c) (referred to as V_{HbA1c}) by using at least three PPG signals with associated with optical signals having one of three different wavelengths λ1, λ2, and λ3. The processing element may determine a ratio of AC to DC (ACRλn) for PPG signals associated with each wavelength (EQ. 1). In embodiments involving use of PPG signals associated with three wavelengths (n=1, 2 and 3), the processing element may determine three AC-to-DC ratios ACR_{λ1}-ACR_{λ3} (EQ. 1a-1c). Subsequently, the processing element may be configured to simultaneously assess a set of linear equations (EQ. 2-4) to determine the blood content of deoxyhemoglobin (V_{HHb}), the blood content of oxyhemoglobin (V_{O2Hb}) and the blood content of glycated hemoglobin (V_{HbA1c}), where k_{AN}, k_{BN}, k_{CN} and cₙ are constants stored in a memory element. Subsequently, the determined blood content of deoxyhemoglobin (V_{HHb}), the blood content of oxyhemoglobin (V_{O2Hb}) and the blood content of glycated hemoglobin (V_{HbA1c}) are used to determine a fractional blood oxygen saturation (Fractional SpO2) (EQ. 5), functional blood oxygen saturation (Functional SpO2) (EQ. 6), and glycated hemoglobin (HbA1c) (EQ. 7).

$${ACR}_{λ n} = {AC}_{λ n} / {DC}_{λ n} \times {c}_{n} [ n = 1 , 2 , 3 , 4 … ] \qquad [ EQ . 1 ]$$

$${ACR}_{λ 1} = {AC}_{λ 1} / {DC}_{λ 1} \times {c}_{1} \qquad [ EQ . 1 a ]$$

$${ACR}_{λ 2} = {AC}_{λ 2} / {DC}_{λ 2} \times {c}_{2} \qquad [ EQ . 1 b ]$$

$${ACR}_{λ 3} = {AC}_{λ 3} / {DC}_{λ 3} \times {c}_{3} \qquad [ EQ . 1 c ]$$

$${ACR}_{λ 4} = {AC}_{λ 4} / {DC}_{λ 4} \times {c}_{4} \qquad [ EQ . 1 d ]$$

$${ACR}_{λ 1} = {V}_{HHb} {k}_{A 1} + {V}_{O 2 Hb} {k}_{B 1} + {V}_{HbA 1 c} {k}_{C 1} \qquad [ EQ . 2 ]$$

$${ACR}_{λ 2} = {V}_{HHb} {k}_{A 2} + {V}_{O 2 Hb} {k}_{B 2} + {V}_{HbA 1 c} {k}_{C2} \qquad [ EQ . 3 ]$$

$${ACR}_{λ 3} = {V}_{HHb} {k}_{A 3} + {V}_{O 2 Hb} {k}_{B3} + {V}_{HbA 1 c} {k}_{C 3} \qquad [ EQ . 4 ]$$

$$Fractional SpO 2 = {V}_{O 2 Hb} / ( {V}_{HHb} + {V}_{O 2 Hb} + {V}_{HbA 1 c} ) \qquad [ EQ . 5 ]$$

$$Functional SpO 2 = {V}_{O 2 Hb} / ( {V}_{HHb} + {V}_{O 2 Hb} ) \qquad [ EQ . 6 ]$$

$$HbA 1 c = {V}_{HbA 1 c} / ( {V}_{HHb} + {V}_{O 2 Hb} + {V}_{HbA 1 c} ) \qquad [ EQ . 7 ]$$

The processing element is communicatively coupled with the memory element that is configured to store absorption constants for deoxyhemoglobin blood content (V_{HHb}), oxyhemoglobin blood content (V_{O2Hb}) and glycated hemoglobin blood content (V_{HbA1c}). In embodiments, each absorption constant is associated with different optical signal wavelengths λ1, λ2 and λ3. For example, the memory element may be configured to store different absorption constants for deoxyhemoglobin blood content (V_{HHb}) for the first wavelength (λ1), the second wavelength (λ1) and the third wavelength (λ3), which are k_{A1}, k_{A2} and k_{A3}, respectively. Similarly, the memory element may be configured to store different absorption constants for oxyhemoglobin blood content (V_{O2Hb}) for the first wavelength (λ1), the second wavelength (λ1) and the third wavelength (λ3), which are k_{B1}, k_{B2} and k_{B3}, respectively. Similarly, the memory element may be configured to store different absorption constants for glycated hemoglobin blood content (V_{HbA1c}) for the first wavelength (λ1), the second wavelength (λ1) and the third wavelength (λ3), which are k_{C1}, k_{C2} and k_{C3}, respectively.

In embodiments, absorption constants kXn (X=A, B, C; n=1, 2, 3, . . . ) include, in addition to absorption characteristics of blood components at respective wavelengths, other optical characteristics of blood components at respective wavelengths λ1, λ2 and λ3. For example, constants kXn may combine absorption and scattering characteristics of blood components at respective wavelengths. In embodiments, constants kXn may also account for optical characteristics other than absorption and scattering.

In embodiments, the memory element stores a signal path factor (cₙ) corresponding to a signal path between an optical transmitter from which an optical signal is output and an optical receiver that receives the optical signal modulated by the skin of the user for different optical signal wavelengths λ1, λ2 and λ3. For example, the memory element may store different signal path factors for the first wavelength (λ1), the second wavelength (λ2) and the third wavelength (λ3), which are c₁, c₂ and c₃, respectively. Alternatively, the memory element may store a common (the same) signal path factor for the first wavelength (λ1), the second wavelength (λ2) and the third wavelength (λ3), which are c₁, c₂ and c₃, respectively. In some embodiments, the signal path factors (C₁-C₃) are predetermined and may be selected by the processing element based on a classification of an individual user based on a model that is associated with classifications of an individual user based on health characteristics, such as age, gender, weight, height, physical condition (e.g., in good health, pulmonary conditions, etc.), fitness class (i.e., overall physical fitness level) and/or metabolic health (e.g., HbA1c level, glucose level, etc.), or an average level of one of more of the foregoing metrics, for the user. In other embodiments, the processing element may dynamically determine the signal path factors (C₁-C₃) for each determination of glycated hemoglobin blood level (HbA1c), fractional blood oxygen saturation level (Functional SpO2) or functional blood oxygen saturation level (Functional SpO2). For instance, the memory element may store a distance between each optical transmitter and each optical receiver and the processing element may obtain and use the distance of the signal path between an optical transmitter from which an optical signal is output and an optical receiver that receives the optical signal modulated by the skin of the user and the optical signal's wavelength (λ1, λ2 or λ3) to determine each signal path factor (C₁-C₃). Similarly, in embodiments, the processing element may determine an “effective” distance of a signal path (between an optical transmitter from which an optical signal is output and an optical receiver that receives the optical signal modulated by the skin of the user) that corresponds to a sum of the lengths of one or more segments of the signal path that are associated with an optical signal passing through pulsatile tissue or skin of a user (as portions of the signal path include non-pulsatile tissue which is outside of pulsatile vasculature and pulsatile tissue including vasculature such as arteries, arterioles or capillaries). Once the processing element has determined an “effective” distance for one or more signal paths, the processing element may store the determined “effective” distances into the memory element for use with determining the signal path factors (C₁-C₃). Additionally, as optical signals of different wavelengths (λ1, λ2 and λ3) may pass through (penetrate) different depths or reach different regions of the user's skin or tissue and originate from optical transmitters that are positioned at slightly different positions of (within) an optical transmitter array, the characteristics of the signal path for each optical signal may be different. In embodiments, the processing element may be configured in such embodiments to dynamically determine the signal path factors (C₁-C₃) for each determination of glycated hemoglobin blood level (HbA1c), fractional blood oxygen saturation level (Functional SpO2) or functional blood oxygen saturation level (Functional SpO2) to account for such differences in the signal path of each optical signal and store the determined signal path factors (c₁-c₃) in the memory element. Alternatively, it is to be understood that use of optical signals having a fourth wavelength (λ4) or several additional wavelengths (e.g., λ4, λ5, etc.) may enable the processing element to dynamically determine the signal path factors (C₁-C₃) similar to the techniques described herein for the processing element using optical signals having one of three different wavelengths to determine within the user's hemoglobin (Hb) a volume of deoxyhemoglobin blood content (V_{HHb}), a volume of oxyhemoglobin blood content (V_{O2Hb}) and a volume of glycated hemoglobin blood content (V_{HbA1c}), as described above. In other words, the processing element may utilize predetermined absorption constants k_{A1}, k_{A2} and k_{A3} for deoxyhemoglobin blood content (V_{HHb}), oxyhemoglobin blood content (V_{O2Hb}) and glycated hemoglobin blood content (V_{HbA1c}), respectively, to determine one or more signal path factors (c₁-c₃) as well as deoxyhemoglobin blood content (V_{HHb}), oxyhemoglobin blood content (V_{O2Hb}) and glycated hemoglobin blood content (V_{HbA1c}). For instance, in embodiments in which the processing element determines a first signal path factor (c₁) dynamically, the processing element may utilize a relationship between a first signal path factor (c₁) and a second signal path factor (c₂) and a third signal path factor (c₃) for a plurality of wavelength groups (e.g., a wavelength length including λ1, λ2 and λ3, a wavelength length including λ1, λ2 and λ4, a wavelength length including λ4, λ5 and λ6; etc.) stored in the memory element to estimate the second signal path factor (c₂) and the third signal path factor (c₃).

In embodiments, the processing element may identify certain equivalency relationships (e.g., EQ. 1a and EQ. 2, EQ. 1b and EQ. 3, EQ. 1c and EQ. 4, etc.) and access from the memory element stored absorption constants for deoxyhemoglobin blood content (k_{A1}-k_{A3}), oxyhemoglobin blood content (k_{B1}-k_{B3}) and absorption constants for glycated hemoglobin blood content (k_{C1}-k_{C3}) to determine within the user's hemoglobin (Hb) a volume of deoxyhemoglobin blood content (V_{HHb}), a volume of oxyhemoglobin blood content (V_{O2Hb}) and a volume of glycated hemoglobin blood content (V_{HbA1c}).

The processing element can then determine a glycated hemoglobin blood level (HbA1c) for the user based on a ratio of the determined glycated hemoglobin blood content (V_{COHb}) to a sum of the determined the deoxyhemoglobin blood content (V_{HHb}), the determined oxyhemoglobin blood content (V_{O2Hb}) and the determined glycated hemoglobin blood content (V_{HbA1c}), as shown in EQ. 7.

In embodiments involving the memory element storing signal path factors (cₙ), once the processing element has obtained from the memory element a stored signal path factor for different optical signal wavelengths (c₁-c₃ for λ1-λ3 or c₁-c₄ for λ1-λ4), the processing element is configured to utilize the signal path factors (C₁-C₃ or C₁-C₄) to determine within the user's hemoglobin (Hb) a volume of deoxyhemoglobin blood content (V_{HHb}), a volume of oxyhemoglobin blood content (V_{O2Hb}) and a volume of glycated hemoglobin blood content (V_{HbA1c}). In other embodiments, the processing element dynamically determines signal path factors for different optical signal wavelengths (c₁-c₃ for λ1-λ3 or c₁-c₄ for λ1-λ4) as part of determining a volume of deoxyhemoglobin blood content (V_{HHb}), a volume of oxyhemoglobin blood content (V_{O2Hb}) and a volume of glycated hemoglobin blood content (V_{HbA1c}), or once these volumes (V_{HHb}, V_{O2Hb} and V_{HbA1c}) have been determined. The processing element is configured to determine a glycated hemoglobin blood level (HbA1c), fractional blood oxygen saturation level (Functional SpO2) or functional blood oxygen saturation level (Functional SpO2) for the user based on the determined volumes of deoxyhemoglobin blood content (V_{HHb}), oxyhemoglobin blood content (V_{O2Hb}) and glycated hemoglobin blood content (V_{HbA1c}).

As the fractional blood oxygen saturation (Functional SpO2) and the functional blood oxygen saturation (Functional SpO2) are determined using Equations 5 and 6, respectively, such determined blood oxygen saturation levels may be more accurate than conventional blood oxygen saturation determinations that may be based on a model that is associated with classifications of an individual user based on health characteristics, such as age, gender, weight, height, physical condition (e.g., in good health, pulmonary conditions, etc.), fitness class (i.e., overall physical fitness level) and/or metabolic health (e.g., HbA1c level, glucose level, etc.), or an average level of one of more of the foregoing metrics.

In embodiments, a fifth PPG signal with a fifth optical signal wavelength λ5 is used to determine a heart rate for the user and a sixth PPG signal with a sixth optical signal wavelength λ6 is used as a cardiac signal reference.

In embodiments, in addition to determining glycated hemoglobin blood content (V_{HbA1c}), oxyhemoglobin blood content (V_{O2Hb}) and deoxyhemoglobin blood content (V_{HHb}), the processing element of the electronic device may be configured to also determine blood content of one or more dyshemoglobin, such as carboxyhemoglobin (V_{COHb}), methemoglobin (V_{MHb}), sulfhemoglobin (V_{SHb}), and hematocrit (V_{SpHb}) and a blood level of each. For example, the processing element may determine glycated hemoglobin blood content (V_{HbA1c}), oxyhemoglobin blood content (V_{O2Hb}) and deoxyhemoglobin blood content (V_{HHb}) as well as carboxyhemoglobin blood content (V_{COHb}), methemoglobin blood content (V_{MHb}) and sulfhemoglobin blood content (V_{SHb}) and a corresponding a carboxyhemoglobin level (COHb) (volume of carboxyhemoglobin blood content (V_{COHb}) relative to the volume all determined hemoglobin (Hb)), a methemoglobin level (MHb) (volume of methemoglobin blood content (V_{MHb}) relative to the volume all determined hemoglobin (Hb)) and a sulfhemoglobin level (SHb) (volume of sulfhemoglobin blood content (V_{SHb}) relative to the volume all determined hemoglobin (Hb)). In embodiments, the processing element may control an optical transmitter to output an optical signal having a fourth wavelength that may be utilized to determine blood content for a fourth hemoglobin (Hb), such as such as carboxyhemoglobin (V_{COHb}), methemoglobin (V_{MHb}), sulfhemoglobin (V_{SHb}), which correspond to a carboxyhemoglobin level (COHb), a methemoglobin level (MHb) and a sulfhemoglobin level (SHb) for the user, respectively. For example, the processing element may control an optical transmitter array to output at least four PPG signals with associated with optical signals having one of four different wavelengths λ1, λ2, λ3 and λ4 to determine at least one of a carboxyhemoglobin level (COHb), a methemoglobin level (MHb) or a sulfhemoglobin level (SHb) for the user. The processing element may be configured to determine a ratio of AC-to-DC ratios (ACRλn) for PPG signals associated with each of the four wavelength (as shown above in EQ. 1) and simultaneously assess a set of linear equations (EQ. 8-11) to determine the blood content of deoxyhemoglobin (V_{HHb}), the blood content of oxyhemoglobin (V_{O2Hb}), the blood content of glycated hemoglobin (V_{HbA1c}) and the blood content of carboxyhemoglobin (V_{COHb}), where k_{AN}, k_{BN}, k_{CN}, k_{DN} and cₙ are constants stored in the memory element.

$${ACR}_{λ 1} = {V}_{HHb} {k}_{A 1} + {V}_{O 2 Hb} {k}_{B 1} + {V}_{HbA 1 c} {k}_{C 1} + {V}_{COHb} {k}_{D 1} \qquad [ EQ . 8 ]$$

$${ACR}_{λ 2} = {V}_{HHb} {k}_{A 2} + {V}_{O 2 Hb} {k}_{B 2} + {V}_{HbA 1 c} {k}_{C 2} + {V}_{COHb} {k}_{D 2} \qquad [ EQ . 9 ]$$

$${ACR}_{λ 3} = {V}_{HHb} {k}_{A 3} + {V}_{O 2 Hb} {k}_{B 3} + {V}_{HbA 1 c} {k}_{C 3} + {V}_{COHb} {k}_{D 3} \qquad [ EQ . 10 ]$$

$${ACR}_{λ 4} = {V}_{HHb} {k}_{A 4} + {V}_{O 2 Hb} {k}_{B 4} + {V}_{HbA 1 c} {k}_{C 4} + {V}_{COHb} {k}_{D 4} \qquad [ EQ . 11 ]$$

$$COHb = {V}_{COHb} / ( {V}_{HHb} + {V}_{O 2 Hb} + {V}_{HbA 1 c} + {V}_{COHb} ) \qquad [ EQ . 12 ]$$

In such an example, the memory element is configured to store absorption constants (k_{AN}-k_{DN}) for deoxyhemoglobin blood content (V_{HHb}), oxyhemoglobin blood content (V_{O2Hb}), glycated hemoglobin blood content (V_{HbA1c}) and carboxyhemoglobin blood content (V_{COHb}) for optical signal wavelengths λ1, λ2, λ3 and λ4 and a signal path factor (cₙ) for wavelength λ1, λ2, λ3 and λ4. As with the examples provided above, the processing element may identify certain equivalency relationships (e.g., EQ. 1a and EQ. 8, EQ. 1b and EQ. 9, EQ. 1c and EQ. 10, EQ. Id and EQ. 11, etc.) and access from the memory element stored absorption constants for deoxyhemoglobin blood content (k_{A1}-k_{A4}), oxyhemoglobin blood content (k_{B1}-k_{B4}), absorption constants for glycated hemoglobin blood content (k_{C1}-k_{C4}) and absorption constants for carboxyhemoglobin blood content (k_{D1}-k_{D4}) to determine within the user's hemoglobin (Hb) a volume of deoxyhemoglobin blood content (V_{HHb}), a volume of oxyhemoglobin blood content (V_{O2Hb}), a volume of glycated hemoglobin blood content (V_{HbA1c}) and carboxyhemoglobin blood content (V_{COHb}). The processing element can then determine a carboxyhemoglobin blood level (COHb) for the user based on a ratio of the determined carboxyhemoglobin blood content (V_{COHb}) to a sum of the determined the deoxyhemoglobin blood content (V_{HHb}), the determined oxyhemoglobin blood content (V_{O2Hb}), the determined glycated hemoglobin blood content (V_{HbA1c}) and the determined carboxyhemoglobin blood content (V_{COHb}), as shown in EQ. 12. It is to be understood that the processing element may determine a methemoglobin level (MHb) or a sulfhemoglobin level (SHb) for the user using a similar technique.

In embodiments, the processing element of the electronic device may identify one or more signal paths having an acceptable signal quality metric, such as signal-to-noise ratio (SNR), for determining physiological information about the user, such as determining a user's pulse or heart rate, the glycated hemoglobin blood level (HbA1c), the fractional blood oxygen saturation level (Functional SpO2), the functional blood oxygen saturation level (Functional SpO2), an estimated stress level, a maximum rate of oxygen consumption (VO2 max), or the like.

Accordingly, the processing element of the electronic device noninvasively determines blood-related and cardiac physiological metrics and information by analyzing one or more PPG signals, such as the PPG signal shown as a waveform in FIG. 2, using pulse spectroscopy techniques to provide optical cardiac monitoring.

In embodiments, similar to conventional electronic devices, the processing element is configured to determine a pulse oximetry indicator, which is equal to a first quotient of the AC value and the DC value at a first optical signal wavelength divided by a second quotient of the AC value and the DC value at a second optical signal wavelength. The indicator may be given by equation EQ. 8, wherein λ1 is the first optical signal wavelength, and λ2 is the second optical signal wavelength:

$$Pulse Oximetry Indicator = \frac{{AC}_{λ 1} / {DC}_{λ 1}}{{AC}_{λ 2} / {DC}_{λ 2}} \qquad [ EQ . 8 ]$$

It is to be understood that any combination of wavelengths could be utilized that enables adequate isolation and separation by the empirical or calculated kAₙ, kBₙ, kCₙ, cₙ constants to accurately determine blood content of deoxyhemoglobin (V_{HHb}), the blood content of oxyhemoglobin (V_{O2Hb}) and the blood content of glycated hemoglobin (V_{HbA1c}). For example, in some embodiments associated with FIG. 5A, λ1 and λ2 are in the “Band 2” and λ3 is in “Band 4.” Similarly, in other embodiments, λ1 and λ2 are in the “Band 2” and λ3 is in “Band 3.” In some embodiments, λ1 is in the “Band 2,” λ2 is in “Band 3,” and λ3 is in “Band 4.” In other embodiments, λ1 is in the “Band 2” and λ2 and λ3 are in one of “Band 3” or “Band 4.”

As the processing element of the electronic device may be configured to determine and present a fractional pulse oximetry level (Fractional SpO2) and a functional pulse oximetry level (Functional SpO2), at least two of the wavelengths used for determining blood content of deoxyhemoglobin (V_{HHb}), the blood content of oxyhemoglobin (V_{O2Hb}) and the blood content of glycated hemoglobin (V_{HbA1c}) may correspond to wavelengths commonly used to determine a blood oxygen saturation level for a user. For example, the processing element may utilize PPG signals generated using optical signals having a first wavelength λ1 in a 620-660 nm band and a third wavelength λ3 in a 850-950 nm band with a second wavelength λ2 in a 660-700 nm band to determine blood content of deoxyhemoglobin (V_{HHb}), the blood content of oxyhemoglobin (V_{O2Hb}) and the blood content of glycated hemoglobin (V_{HbA1c}).

Generally, as seen in FIG. 2, the PPG signal waveform includes an AC component and a DC component. The AC component of the PPG signal waveform oscillates between a local maximum and a local minimum over successive short periods of time. The DC component of the PPG signal waveform may be a moving average of the local maximum and the local minimum over successive short periods of time. In some implementations, the processing element may control a low-pass filter to isolate the DC component or a substantial portion of the DC component. A source of low-frequency AC noise, such as a motion component, may be included in the DC value, causing the PPG signal waveform to move up or down. Some low-frequency AC noise components, having frequencies similar to the cardiac component, modulate the envelope of and/or otherwise distort the cardiac component of the PPG signal. Noise inherent in the transmission of the optical signals through a user's skin or tissue as well as motion and other AC noise components may make the identification and extraction of the cardiac component, such as the one shown in FIG. 4, by the processing element difficult. As discussed above, optical signals of different wavelengths (λ1, λ2 and λ3) may pass through (penetrate) different regions of the user's skin and originate from optical transmitters that are positioned at slightly different positions of (within) an optical transmitter array, which may cause the characteristics of the signal path for each optical signal to be different. In embodiments, the processing element may be configured to dynamically determine the signal path factors (e.g., c₁-c₃) for each determination of glycated hemoglobin blood level (HbA1c), fractional blood oxygen saturation level (Functional SpO2) or functional blood oxygen saturation level (Functional SpO2) to account for such differences in the signal path of each optical signal and store the determined signal path factors (e.g., c₁-c₃) in the memory element.

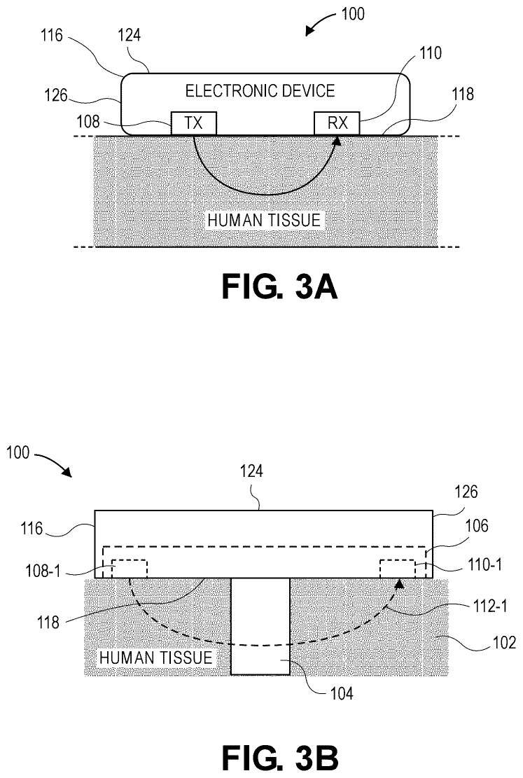

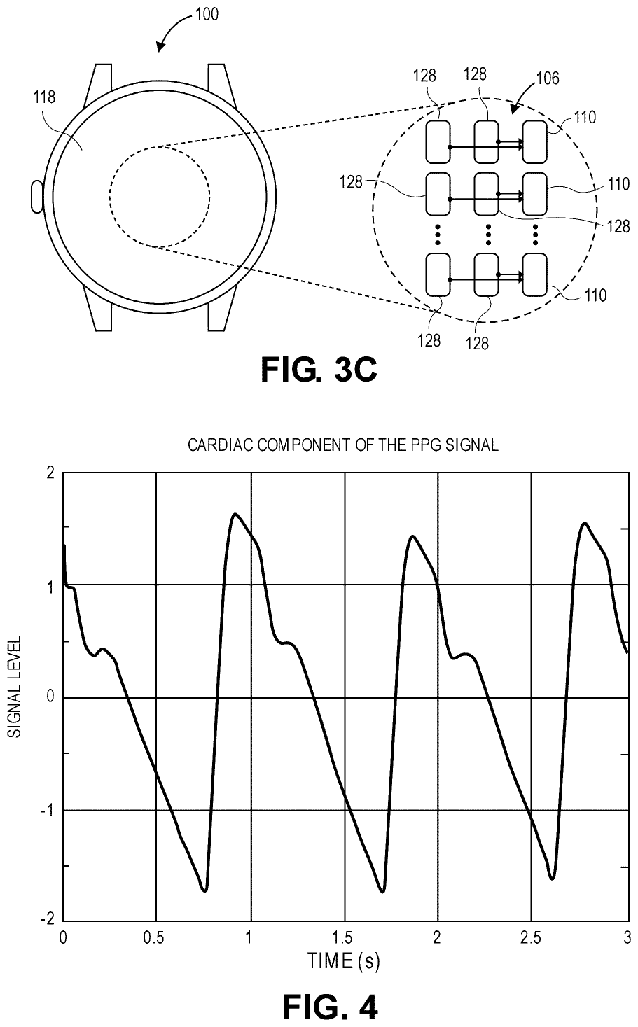

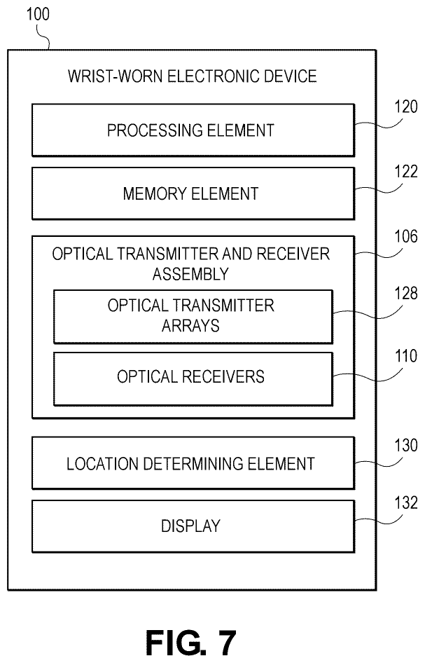

As shown in FIGS. 1 and 3A-3C, the wrist-worn electronic device 100 includes a housing 116, which generally houses or retains other components of the wrist-worn electronic device 100 and may include or be coupled to a wrist band 104. The housing 116 includes a bottom wall 118 incorporating an optical transmitter and receiver assembly 106, which may include a plurality of optical transmitter arrays 128 (each optical transmitter array 128 including one or more optical transmitters 108 (TX)) and optical receivers 110 (RX), an upper surface 124, at least one side wall 126 that bound an internal cavity (not shown in the figures) and the wrist band 104 to attach the wrist-worn electronic device 100 to a wrist of the user 102. As shown in FIG. 7, the wrist-worn electronic device includes a processing element 120, a memory element 122 and a display 132, the processing element 120 configured to determine physiological information about the user, such as the user's heart rate, glycated hemoglobin blood level (HbA1c) and blood oxygen saturation levels (SpO2).

The bottom wall 118 includes a lower, outer surface that contacts the user's wrist while the user is wearing the wrist-worn electronic device 100. The bottom wall 118 may be substantially flat with a slight protrusion that enables the bottom wall 118 to contact a substantial portion of the user's upper wrist. The upper surface 124 opposes the bottom wall 118. In various embodiments, the upper surface 124 may further include an opening that extends from the upper surface 124 to the internal cavity. In some embodiments, such as the exemplary embodiments shown in the figures, the bottom wall 118 of housing 116 may have a round, circular, or oval shape, with a single circumferential side wall 126. In other embodiments, the bottom wall 118 may have a four-sided shape, such as a square or rectangle, or other polygonal shape, with the housing 116 including four or more sidewalls. As seen in FIG. 3C, bottom wall 118 of the housing 116 may include one or more openings in which a plurality of optical transmitter arrays 128, at least some of which include a plurality of optical transmitters 108, and the optical receivers 110 are placed, positioned, or located. The one or more openings within the bottom wall 118 of the housing 116 may be covered by one or more lenses through which the optical signals may be transmitted and received.

Referring to FIG. 3A, a PPG signal corresponds to an optical signal (light) emitted from an optical transmitter 108 (TX) into the user's skin (human tissue) proximate to an optical receiver 110 (RX). The user (wearer) may be any individual who wears the electronic device such that a housing of the electronic device is located proximate to skin of the individual (e.g., worn against the person's wrist, arm, finger, abdomen, leg, etc.). The emitted optical signal penetrates the user's skin with substantial energy to a depth that ranging from tens of microns to several millimeters depending on a variety of criteria, such as the wavelength of transmitted light, distance between the optical transmitter 108 and the optical receiver 110, presence of blood vessels and composition of the user's skin layers. A portion of the optical signal is reflected, or otherwise transferred, from the skin to the optical receiver 110 (RX), typically a photodiode, that generates the PPG signal. The magnitude of the PPG signal is associated with an intensity of the received optical signal (light). The optical signal may be modulated, or otherwise modified, by the flow of blood through the vessels in the path of the optical signal. Specifically, the optical signal is modulated by the blood flow response to the beating of the user's heart, or the cardiac cycle. Thus, the optical signal received by the optical receiver 110 (RX) has been modulated to include a cardiac component corresponding to the user's cardiac characteristics, which are associated with the user's heartbeat. In turn, the PPG signal generated by the optical receiver 110 (RX) includes a cardiac component corresponding to the effect of the user's heartbeat on the flow of blood in the vessels. An example of the cardiac component of the PPG signal is shown in FIG. 4.

The optical transmitter and receiver assembly 106 is located on or within a bottom wall 118 of the wrist-worn electronic device 100 such that it is positioned adjacent to skin of the user 102 when the housing 116 is secured to the user's wrist by wrist band 104. The optical transmitter and receiver assembly 106 includes a plurality of optical transmitters 108 and a plurality of optical receivers 110. The optical transmitter and receiver assembly 106, optical transmitters 108, and optical receivers 110 are each depicted in FIG. 3B by dashed lines because the optical transmitter and receiver assembly 106, optical transmitters 108, and optical receivers 110 are positioned within and/or at the bottom wall 118 of the wrist-worn electronic device 100.

In FIG. 3B, one of the plurality of optical transmitters and one of the plurality of optical receivers are depicted. For example, a first optical transmitter 108-1 is positioned at a first location on the bottom wall 118 and is operable to output a first optical signal that passes through a user's skin. The first optical receiver 110-1 is positioned at a second location on the bottom wall 118 and is operable to receive the first optical signal from the first optical transmitter 108-1 such that the optical signals travel along a first signal path 112-1 from the first optical transmitter 108-1 to the first optical receiver 110-1. The first signal path 112-1 from the first location to the second location is substantially parallel to the arm axis of the user 102. It is to be understood that additional optical signals transmitted by other optical transmitters 108 and received by optical transmitters 110 may travel a substantially similar signal path as the first optical signal.

In some embodiments, an optical transmitter array 128-N includes a first optical transmitter 108-N1 configured by processing element 120 to transmit a first optical signal having a first wavelength, a second optical transmitter 108-N2 configured by processing element 120 to transmit a second optical signal having a second wavelength and a third optical transmitter 108-N3 (not depicted in the Figures) configured by processing element 120 to transmit a third optical signal having a third wavelength, each optical signal separated in time such that the optical transmitter array 128-N is controlled by the processing element 120 to transmit the first optical signal during a first period of time, the second optical signal during a second period of time and the third optical signal during a third period of time. Such time divisional multiplexing (TDM) techniques enable the processing element 120 to control the specific time period (one of a plurality of predetermined time windows) during which each of the plurality of optical transmitters may transmit an optical signal having a particular wavelength into the user's skin. As the number of sequential optical transmissions increase, the duration of time required for all transmissions to occur sequentially increases, which extends the minimum period of time between successive optical signal transmissions in a transmission sequence.

In other embodiments, processing element 120 may utilize techniques to enable the first optical transmitter 108-N1, second optical transmitter 108-N2 and the third optical transmitter 108-N3 (not depicted in the Figures) of optical transmitter array 128-N to transmit a first optical signal having a first wavelength, a second optical signal having a second wavelength and a third optical signal having a third wavelength simultaneously (all three optical signals being transmitted during first time period, a second time period and/or a third time period) or at partially overlapping time periods (one or more of the three optical signals being transmitted during first time period, a second time period and/or a third time period). For instance, the processing element 120 may implement the modulation of each optical signal at different (non-overlapping), predetermined frequencies, signal phases and/or waveforms to enable the multiplexing of a plurality of optical signals. Such modulation techniques enable the processing element 120 to control modulators to generate carrier signals having different frequencies and a plurality of optical transmitters 108 to enable all of the optical transmitters 108 to output optical signals simultaneously either continuously or for a predetermined duration of time. Details about and examples related to such modulation techniques are provided in application Ser. No. 18/637,928, entitled “Use of Frequency Division Multiplexing for Optical Cardiac Signals,” filed on Apr. 17, 2014, which is incorporated by reference in its entirety.

An optical receiver 110-N measures an intensity of the first optical signal, the second optical signal and the third optical signal, and generates a first PPG signal corresponding to the measured intensity of the first optical signal, a second PPG signal corresponding to the measured intensity of the second optical signal and a third PPG signal corresponding to the measured intensity of the third optical signal. The intensity of the first optical signal, the second optical signal and the third optical signal varies in accordance with the amount of blood in the regions of the user's tissue in the signal path 112-N and changes as the blood is moved through the body of the user 102 with each heartbeat. The changing levels of blood in the tissue of the skin of the user 102 proximate to the optical transmitter and receiver assembly 106 along the signal path 112-N results in different intensity of the first, second and third optical signals, respectively. Accordingly, the processing element 120 can determine physiological information for the user 102 based on the first PPG signal, the second PPG signal, the third PPG signal or a combination thereof.

In embodiments, an optical receiver 110-N receives optical signals output by a plurality of optical transmitter arrays 128. For example, the second optical signal may be output from an optical transmitter 108 of a second optical transmitter array 128-2 (not depicted in FIG. 3B) and reflected from the upper layers of skin of the user 102 towards the optical receiver 110-N along a second signal path 112-2 (not depicted in FIG. 3B). Similarly, the third optical signal may be output from an optical transmitter of a third optical transmitter array 128-3 (not depicted in FIG. 3B) and reflected from the upper layers of skin of the user 102 towards the optical receiver 110-N along a third signal path 112-3 (not depicted in FIG. 3B). The optical receiver 110-N measures an intensity of the first optical signal, the second optical signal and the third optical signal, and generates a first PPG signal corresponding to the measured intensity of the first optical signal, a second PPG signal corresponding to the measured intensity of the second optical signal and a third PPG signal corresponding to the measured intensity of the third optical signal. It is to be understood that, in some embodiments, a second optical receiver 110-2 (not depicted in FIG. 3B) may receive one or more of the first, second and third optical signals output by the first transmitter array 128-1, the second transmitter array 128-2 and the third transmitter array 128-3, respectively, with at least one optical signal traveling along a second signal path 112-2 (not depicted in FIG. 3B).

In embodiments, the first optical receiver 110-1 is separated from the first optical transmitter 108-1 such that the first optical signal transmitted from (output by) the first optical transmitter 108-1 travels to the first optical receiver 110-1 along signal path 112-1 that is substantially parallel to the arm axis of the user 102. The arm axis extends along a portion of a length of an arm of the user 102 from an elbow to a hand of that arm. The second optical receiver 110-2 may be separated from the second optical transmitter 108-2 such that the second optical signal transmitted from (output by) the second optical transmitter 108-2 travels to the second optical receiver 110-2 along signal path 112-2 that is substantially parallel to the arm axis of the user 102. Signal path 112-1 is depicted in FIG. 3B using dashed lines because the signal path 112-N are within the upper layers of skin tissue of the user 102 under the bottom wall 118 of the wrist-worn electronic device 100 when the wrist-worn electronic device 100 is worn by the user 102.

In embodiments, a first lens is positioned along bottom wall 118 at the first location over the first optical transmitter 108-1 and a second lens is positioned along bottom wall 118 at the second location over the second optical transmitter 108-2. In such embodiments, if a plurality of optical signals are output by the first optical transmitter 108-1, the plurality of optical signals pass through the first lens and are received by the first optical receiver 110-1. Similarly, if a plurality of optical signals are output by the second optical transmitter 108-2, the plurality of optical signals pass through the second lens and are received by the second optical receiver 110-2. FIG. 3B illustrates one of a plurality of signal paths 112-N that may be substantially parallel to a user's arm axis.

In embodiments, each optical transmitter 108 may include a photonic generator, such as a light-emitting diode (LED), a modulator, a top emitter, an edge emitter, or the like. The photonic generator receives an electrical input signal from the processing element 120 that may be a control signal, such as an electric voltage or electric current that is analog or digital, or data, either of which is indicative of activating or energizing the optical transmitter 108 to output an optical signal having a desired amplitude, frequency, and duration. The photonic generator of each optical transmitter 108 transmits or outputs electromagnetic radiation having a particular wavelength (the optical signal) in the visible light spectrum, which is typically between approximately 400 nanometers (nm) to 700 nm, or in the near infrared spectrum, which is typically between approximately 700 nm to 1,000 nm. In some embodiments, the photonic generator transmits electromagnetic radiation in a wavelength range of 1,000 nm to 1,500 nm. The wavelength of the optical signal is generally determined by, or varies according to, the material from which the photonic generator of each optical transmitter 108 is formed. The optical signal may comprise a sequence of pulses, a periodic or non-periodic waveform, a constant level for a given period of time, or the like, or combinations thereof. In other embodiments, each optical transmitter 108 may include a driver circuit, with electronic circuitry such as amplifier and an optional filter, electrically coupled to the photonic generator. The driver circuit may receive the electrical input signal (control signal) from the processing element 120 and the driver circuit may generate an electric voltage or electric current to the photonic generator, which in turn, outputs the optical signal.

Referring to FIGS. 5A-5D, which are plots that show an absorption coefficient, or level of absorption, of various components of blood, including oxygenated blood, deoxygenated blood, and water, for an optical signal emitted by an optical transmitter. The present application discloses optical “pulse” spectroscopy techniques that may be applied by the processing element 120 to determine blood-related and cardiac physiological metrics and information, such as a user's glycated hemoglobin level (HbA1c), a fractional blood oxygen saturation level (Functional SpO2) or a functional blood oxygen saturation level (Functional SpO2), by utilizing optical signals having at least three different wavelengths that may be selected based on a relationship between the absorption level of an optical signal in oxygenated blood (O2Hb), deoxygenated blood (HHb) and glycated hemoglobin (HbA1c). As shown in FIGS. 5A-5D, the absorption of the optical signal is different across the spectrum of 400-1300 nm for each blood component, such as oxygenated blood, deoxygenated blood or water. As a result, transmitting and receiving an optical signal having a suitable wavelength may enable the processing element 120 to determine information about a user's blood and cardiac condition based on known absorption characteristics of the blood at that wavelength.

As shown in FIG. 5B, processing element 120 may control an optical transmitter to emit an optical signal having a wavelength that is within one of a plurality of ranges (bands) of wavelengths. For example, in embodiments, an optical transmitter may be controlled to emit an optical signal having a wavelength of 550 nm (green light), which is within a band labeled “heart rate,” and the processing element 120 may utilize a PPG signal generated by an optical receiver based on received reflections of the optical signal to determine a user's heart rate. Similarly, the processing element 120 may control a first optical transmitter to emit a first optical signal having a wavelength of 630 nm (red light), which is within a band labeled “λ1,” a second optical transmitter to emit a second optical signal having a wavelength of 660 nm (red light), which is within a band labeled “22,” and/or a third optical transmitter to emit a third optical signal having a wavelength of 930 nm (infrared light that is not visible), which is within a band labeled “23,” and utilize PPG signals generated by one or more optical receivers based on received reflections of the 630 nm, 660 nm and 940 nm optical signals to determine a user's glycated hemoglobin level (HbA1C), fractional blood oxygen saturation level (Functional SpO2) or functional blood oxygen saturation level (Functional SpO2).

Similarly, as shown in FIG. 5C, in some embodiments, the processing element 120 may utilize PPG signals generated using optical signals having a first wavelength λ1 in a 660-700 nm band and a second wavelength λ2 in a 800-900 nm band for determining blood oxygen saturation levels and PPG signals generated using optical signals having the first wavelength λ1, the second wavelength λ2 and a third wavelength λ3 in a 950-1020 nm band to determine blood content of deoxyhemoglobin (V_{HHb}), the blood content of oxyhemoglobin (V_{O2Hb}) and the blood content of glycated hemoglobin (V_{HbA1c}), which may enable the processing element 120 to determine a user's glycated hemoglobin (HbA1C), fractional blood oxygen saturation level (Functional SpO2) or functional blood oxygen saturation level (Functional SpO2). The processing element 120 may utilize PPG signals generated using optical signals having a fourth wavelength in a 530-580 nm band to determine a heart rate for the user.

As shown in FIG. 5D, the processing element 120 may utilize PPG signals generated using optical signals having a first wavelength λ1 in a “Pulse Ox 1” band between 630-660 nm, a second wavelength λ2 in a “Pulse Ox 2” band 850-940 nm and a third wavelength λ3 in a 660-700 nm band to determine blood content of deoxyhemoglobin (V_{HHb}), the blood content of oxyhemoglobin (V_{O2Hb}) and the blood content of glycated hemoglobin (V_{HbA1c}), which may enable the processing element 120 to determine a user's glycated hemoglobin level (HbA1C), fractional blood oxygen saturation level (Functional SpO2) or functional blood oxygen saturation level (Functional SpO2). The processing element 120 may utilize PPG signals generated using optical signals having a fourth wavelength in a 520-580 nm band to determine a heart rate for the user.

As stated above, it is to be understood that the boundaries of each wavelength band for the optical signals generated by the optical transmitters could change and implementations in accordance with those variations would be embodiments hereof. In some of the example embodiments provided above, the processing element 120 may control a plurality of optical transmitters that output optical signals (light) each having a desired wavelength that corresponds to a certain absorption level for oxygenated and deoxygenated blood. For example, a first optical transmitter may be configured to output optical signals at a first wavelength in a 500 nm-600 nm band having a high absorption level and visible to the human eye as the color green for use by the processing element 120 with determining a heart rate for the user. In embodiments, a second and a third optical transmitter may be configured to output optical signals at a second wavelength and a third wavelength, respectively, between 600 nm and 1000 nm for use by the processing element 120 with determining a blood oxygen saturation (SpO2) level for the user (in some embodiments, either or both of the second wavelength and the third wavelength may be between 1000 nm and 2000 nm). A fourth optical transmitter may be configured to output optical signals at a fourth wavelength between 600 nm and 1000 nm for use by the processing element 120, in combination with the optical signals generated having the second wavelength and the third wavelength, with the determination of a glycated hemoglobin level (HbA1c), a fractional blood oxygen saturation level (Functional SpO2) or a functional blood oxygen saturation level (Functional SpO2) for the user based on three corresponding PPG signals (in some embodiments, the fourth wavelength may be between 1000 nm and 2000 nm). The optical signals generated having a wavelength between 600 nm and 1000 nm (or between 1000 nm and 2000 nm) penetrate into the deeper (lower) layers of the user's skin tissue in which glycated hemoglobin (HbA1c) may pass through blood vessels of the user. Optical signals having a wavelength between 600 nm-700 nm may be visible to the human eye as the color red and optical signals having a wavelength between 700 nm-1000 nm (or between 1000 nm and 2000 nm) may be invisible to the human eye as near-infrared or infrared signals. In embodiments, a fifth optical transmitter may be configured to output optical signals at a fifth wavelength between 600 nm and 1000 nm (or between 1000 nm and 2000 nm for use by the processing element 120 as a cardiac signal reference).

Accordingly, in embodiments, the wrist-worn electronic device 100 may include optical transmitters 108 (TX) that emit an optical signal having a wavelength that is within one of a plurality of ranges (bands) of wavelengths labeled for use with simultaneously determining a heart rate, a glycated hemoglobin level (HbA1c), a fractional blood oxygen saturation level (Functional SpO2) or a functional blood oxygen saturation level (Functional SpO2) for the user. The processing element 120 may determine blood content of deoxyhemoglobin (V_{HHb}), the blood content of oxyhemoglobin (V_{O2Hb}) and the blood content of glycated hemoglobin (V_{HbA1c}), glycated hemoglobin level (HbA1c), fractional pulse oximetry (Fractional SpO2), and functional pulse oximetry (Functional SpO2) for a user at any time. For instance, the processing element 120 may make the abovementioned determinations multiple times during the user's sleep (typically overnight), sedentary periods (e.g., while a user is sitting in a chair) or at any time desired by a user (e.g., at a scheduled or preferred time, in response to a user input, etc.). In embodiments, the processing element 120 may be configured to determine one or more of these levels when the wrist-worn electronic device 100 is determined to have minimal motion or is substantially motionless as motion may be a potential source of error for determinations made using PPG signals. In such embodiments, the wrist-worn electronic device 100 may include an accelerometer communicatively coupled with the processing element 120 in order for the processing element 120 to assess whether the housing 116 is moving or substantially motionless.

For a user's glycated hemoglobin level (HbA1c), which changes at a much slower rate than the user's blood oxygen saturation level (as the latter can change rapidly as the concentration of oxygenated hemoglobin changes over a series of breathing cycles), the processing element 120 can determine and adjust (update) the user's glycated hemoglobin level (HbA1c) on a weekly or a monthly basis, when desired by the user using a user interface (e.g., touch screen, pushbuttons, etc.) or as otherwise configured to determine the user's glycated hemoglobin level (HbA1c). Accordingly, the processing element 120 can store determined glycated hemoglobin levels (HbA1c) in the memory element 122 and implement techniques to determine a current glycated hemoglobin level (HbA1c) with higher accuracy, which may include identifying determined glycated hemoglobin levels (HbA1c) that are outliers, which may be removed or have a lower weighting applied to them.

Similarly, for a user's determined fractional pulse oximetry level (Fractional SpO2) and determined functional pulse oximetry level (Functional SpO2), which typically change at a much faster rate than the user's glycated hemoglobin level (HbA1c), the processing element 120 can determine and adjust (update) the user's fractional pulse oximetry level (Fractional SpO2) and functional pulse oximetry level (Functional SpO2) on a daily or weekly basis, when desired by the user using a user interface (e.g., touch screen, pushbuttons, etc.) or as otherwise configured to determine the user's fractional pulse oximetry level (Fractional SpO2) and functional pulse oximetry level (Functional SpO2). Accordingly, the processing element 120 can store determined fractional pulse oximetry level (Fractional SpO2) and determined functional pulse oximetry level (Functional SpO2) in the memory element 122 and implement techniques to determine a current fractional pulse oximetry level (Fractional SpO2) and a current functional pulse oximetry level (Functional SpO2) with higher accuracy, which may include identifying determined levels that are outliers, which may be removed or have a lower weighting applied to them.

The processing element 120 may control the display 132 to present the determined glycated hemoglobin level (HbA1c), the determined fractional pulse oximetry level (Fractional SpO2) and the determined functional pulse oximetry level (Functional SpO2) to the user or general feedback on an overall health determined for the user based on the glycated hemoglobin level (HbA1c). Users that may be prediabetic or diabetic can make pharmacological, diet, exercise and lifestyle changes based on that information to improve or maintain his or her overall health.

The memory element 122 may store one or more computer-executable instructions that, when executed by the processing element 120, utilize a PPG signal associated with an optical signal having a wavelength in a band identified in FIGS. 5A-5D. The processing element 120, electronically coupled to the memory element 122, may determine the user's glycated hemoglobin level (HbA1c), fractional pulse oximetry (Fractional SpO2) or functional pulse oximetry (Functional SpO2). In embodiments, the memory element 122 may store a plurality of glycated hemoglobin levels (HbA1c), fractional pulse oximetry levels (Fractional SpO2) and functional pulse oximetry (Functional SpO2) and health and/or physiological characteristics to determine and present one or more recommendations to the user on display 132. Health characteristics may include age, gender, weight, height, physical condition (e.g., in good health, pulmonary conditions, etc.), and fitness class (i.e., overall physical fitness level). Physiological characteristics may include, but are not limited to, a heartbeat, heart rate, heart-rate variability, speed, distance traveled, calculating calories burned, body temperature, blood pressure, stress intensity level, body energy level, and the like.

Generally, the wrist-worn electronic device 100 contains an optical transmitter 108 that is positioned at a first location on the bottom wall 118 and is operable to output a plurality of optical signals, having at least three wavelengths that pass through the skin of a user 102, and an optical receiver 110 that is positioned at a second location on the bottom wall 118 and operable to receive the optical signals from the optical transmitter 108 such that the optical signals travel along a signal path 112 from the optical transmitter 108 to the optical receiver 110. The signal path 112 from the first location to the second location may be substantially parallel to an arm axis of the user 102. In embodiments, the wrist-worn electronic device 100 includes a plurality of optical transmitters 108 and a plurality of optical receivers 110 forming two or more signal paths 112 that may be oriented to be substantially parallel to an arm axis when the wrist-worn electronic device 100 is worn by a user 102.

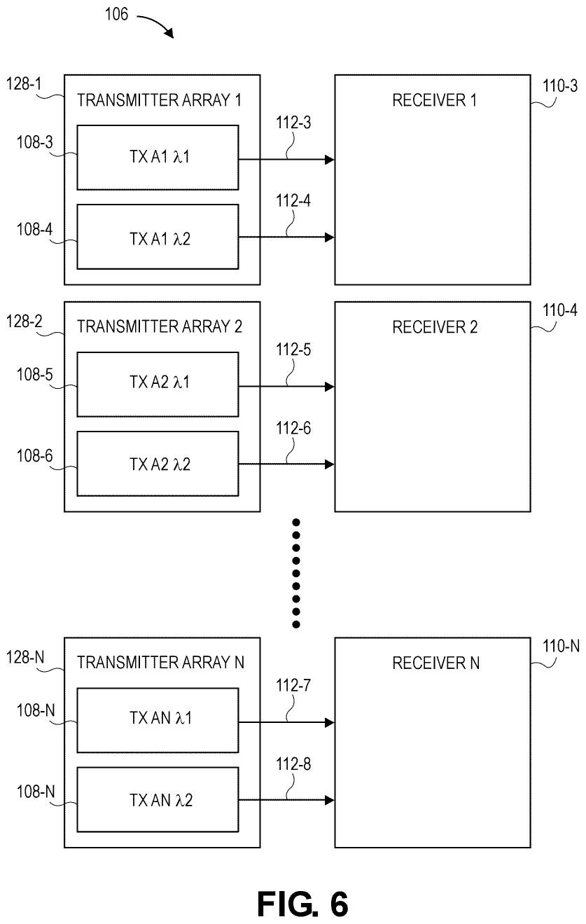

FIG. 6 is a schematic view of a first embodiment of an optical transmitter and receiver assembly 106 having a plurality of optical transmitter arrays 128 and optical receivers 110 illustrating signal paths of the optical signal transmitted by each of the optical transmitters 108 and received by one of a plurality of optical receivers 110. The optical transmitter and receiver assembly 106 includes a plurality of optical transmitter arrays 128-1, 128-2, 128-N and a plurality of corresponding optical receivers 110-3, 110-4, 110-N. Each optical transmitter array 128 includes a plurality of optical transmitters 108 in the same packaging. For example, the first optical transmitter array 128-1 includes a first optical transmitter 108-3 (TX A1 λ1) and a second optical transmitter 108-4 (TX A1 λ2), the second optical transmitter array 128-2 includes a first optical transmitter 108-5 (TX A2 λ1) and a second optical transmitter 108-6 (TX A2 λ2), and an Nth optical transmitter array 128-N includes a first optical transmitter 108-N1 (TX AN λ1) and a second optical transmitter 108-N2 (TX AN λ2). The first optical transmitter 108-3 (TX A1 λ1) of the first optical transmitter array 128-1 is configured to output (emit) a first optical signal having a first wavelength and the second optical transmitter 108-4 (TX A1 λ2) of the first optical transmitter array 128-1 is configured to output (emit) a second optical signal having a second wavelength. It is to be understood that, although FIG. 6 depicts each optical transmitter array 128 having only two optical transmitters 108, each optical transmitter array 128 may incorporate at least a third optical transmitter 108 in order to output (emit) optical signals at a third wavelength. In other words, each optical transmitter array 128-1-128-N may include a third optical transmitter 108 that outputs optical signals having a third wavelength (λ3).

The first optical receiver 110-3 (Receiver 1) is positioned at a location on the bottom wall 118 of the housing 116 that is separated from the first optical transmitter 108-3 (TX A1 λ1) of the first optical transmitter array 128-1 such that the first optical signal output by the first optical transmitter 108-3 (TX A1 λ1) travels along a first signal path 112-3, which may be substantially parallel to an arm axis of a user from the first optical transmitter 108-3 (TX A1 λ1) to first optical receiver 110-3 (Receiver 1). The location of the first optical receiver 110-3 (Receiver 1) is also separated from second optical transmitter 108-4 (TX A1 λ2) such that the second optical signal output by second optical transmitter 108-4 (TX A1 λ2) travels along a second signal path 112-4, which may be substantially parallel to the arm axis of the user from transmitter 108-4 (TX A1 λ2) to the first optical receiver 110-3 (Receiver 1). In some embodiments, the processing element 120 is configured to control the first optical transmitter 108-3 (TX A1 λ1), the second optical transmitter 108-4 (TX A1 λ2) and a third optical transmitter (not depicted in FIG. 6) associated with a third wavelength (λ3) to output the first optical signal, the second optical signal and the third optical signal, respectively, at predetermined times, which may cause the optical signals to be transmitted sequentially, simultaneously or during partially overlapping times. Similarly, the processing element 120 may control the second transmitter array 128-2 to cause the first optical transmitter 108-5 (TX A2 λ1) of the second optical transmitter array 128-2 to output (emit) an optical signal having a first wavelength along a first signal path 112-5 to second optical receiver 110-4 (Receiver 2), the second optical transmitter 108-6 (TX A2 λ2) to output (emit) an optical signal having a second wavelength along a second signal path 112-6 to second optical receiver 110-4 (Receiver 2) and a third optical transmitter 108 to output (emit) an optical signal having a third wavelength along a signal path 112 to the second optical receiver 110-4 (Receiver 2) at predetermined times, which may cause the optical signals to be transmitted sequentially, simultaneously or during partially overlapping times. The same functionality applies to the Nth optical transmitter array 128-N, which may be controlled by the processing element 120 to cause first optical transmitter 108-N1 (TX AN λ1) to output (emit) an optical signal having a first wavelength along a first signal path 112-7 to the Nth optical receiver 110-N(Receiver N), the second optical transmitter 108-N2 (TX AN λ2) to output (emit) an optical signal having a second wavelength along a second signal path 112-8 to the Nth optical receiver 110-N(Receiver N), and a third optical transmitter 108 to output (emit) an optical signal having a third wavelength along a signal path 112 to the Nth optical receiver 110-N(Receiver N) at predetermined times, which may cause the optical signals to be transmitted sequentially, simultaneously or during partially overlapping times.

An exemplary Nth optical transmitter array 128-N may include a first optical transmitter 108 configured or operable to output an optical signal having a first wavelength (λ1), a second optical transmitter 108 configured or operable to output an optical signal having a second wavelength (λ2) and a third optical transmitter 108 configured or operable to output an optical signal having a third wavelength (λ3). In some embodiments, the first wavelength (λ1) may range from approximately 630 nm to approximately 700 nm, the second wavelength (λ2) may range from approximately 700 nm to approximately 800 nm and the third wavelength (λ3) may range from approximately 800 nm to approximately 1,000 nm. In other embodiments, the first wavelength (λ1) may range from approximately 620 nm to approximately 650 nm, the second wavelength (λ2) may range from approximately 650 nm to approximately 700 nm and the third wavelength (λ3) may range from approximately 760 nm to approximately 950 nm. In other embodiments, the first wavelength (λ1) may be between approximately 620 nm and approximately 650 nm, the second wavelength (λ2) may be between approximately 650 nm and approximately 700 nm and the third wavelength (λ3) may be between approximately 800 nm and approximately 850 nm.

In some embodiments, optical transmitter arrays 128-1 through 128-BN may include a plurality of optical transmitters 108 that output optical signals having four wavelengths. For instance, optical transmitter arrays 128-1 through 128-BN may include optical transmitters 108 that output optical signals having a first wavelength in band of 620-650 nm, a second wavelength in band of 650-700 nm, a third wavelength in band of 700-860 nm, and a fourth wavelength in band of 900-1,000 nm. In other configurations, optical transmitter arrays 128-1 through 128-BN may include optical transmitters 108 that output optical signals having two wavelengths in band of 630-700 nm, a third wavelength in band of 700-840 nm, and a fourth wavelength in band of 840-1,025 nm.

Similar to signal path 112-1 shown in FIG. 3B, signal paths 112-2, 112-3, 112-4, 112-5, 112-6, 112-7, 112-8 are also formed within and pass through the skin tissue of a wrist of the user 102 around which the housing 116 is secured using wrist band 104. Within the tissue, the optical signals encounter permanent and/or pulsatile cardiovascular wrist structures such as arterioles and capillaries through which the optical signals modulate and pass through to an optical receiver, which generates a PPG signal that is associated with its signal path 112. Each optical transmitter array 128 may contain a plurality of optical transmitters 108 that are substantially equidistant to an optical receiver 110. For instance, the first optical transmitter 108-3 (TX A1 λ1) and the second optical transmitter 108-4 (TX A1 λ2) within the first optical transmitter array 128-1 may be positioned adjacent to one another and signal paths 112-3, 112-4 to the first optical receiver 110-3 (Receiver 1) may substantially overlap in the sensing (e.g., skin) plane.

In embodiments, one or more of optical arrays 128-3, 128-4 and 128-BN may contain a single optical transmitter 108 having a fourth wavelength (λ4) below 600 nm, which is associated with light visual appearing the color green. The processing element 120 may be configured to determine a heartrate for the user based on output optical signals having a wavelength between approximately 540 nm and approximately 580 nm.

FIG. 7 is a block diagram of the wrist-worn electronic device 100. The wrist-worn electronic device 100 includes the processing element 120, the memory element 122, the optical transmitter and receiver assembly 106, a location determining element 130 and the display 132. As described above, each optical transmitter and receiver assembly 106 may include a plurality of optical transmitters 108 and a plurality of optical receivers 110.

The processing element 120 provides processing functionality for the optical transmitter and receiver assembly 106 and can include any number of processing elements, micro-controllers, circuitry, field programmable gate array (FPGA) or other processing systems, and resident or external memory for storing data, executable code, and other information. The processing element 120 is not limited by the materials from which it is formed or the processing mechanisms employed therein and, as such, can be implemented via semiconductor(s) and/or transistors (e.g., using electronic integrated circuit (IC) components), and so forth.

The processing element 120 can execute one or more software programs embodied in a non-transitory computer readable medium (e.g., memory element 122) that implement techniques described herein including receiving a first PPG signal from optical receivers 110. The processing element may be configured to utilize the PPG signals, which may be stored in memory element 122 or received from the optical receivers 110, to determine physiological information about the user.

In some embodiments, the processing element 120 can determine, based on a plurality of determined AC-to-DC ratios, the blood content of deoxyhemoglobin (V_{HHb}), the blood content of oxyhemoglobin (V_{O2Hb}) and the blood content of glycated hemoglobin (V_{HbA1c}). The processing element 120 can also determine a glycated hemoglobin blood level (HbA1c) for the user based on a ratio of the determined glycated hemoglobin blood content (V_{HbA1c}) to a sum of the determined the deoxyhemoglobin blood content (V_{HHb}), the determined oxyhemoglobin blood content (V_{O2Hb}) and the determined glycated hemoglobin blood content (V_{HbA1c}). In embodiments, the processing element 120 can also determine a fractional blood oxygen saturation level (Fractional SpO2) for the user based on a ratio of the determined oxyhemoglobin blood content (V_{O2Hb}) to a sum of the determined the deoxyhemoglobin blood content (V_{HHb}), the determined oxyhemoglobin blood content (V_{O2Hb}) and the determined glycated hemoglobin blood content (V_{HbA1c}). In embodiments, the processing element 120 can also determine a functional blood oxygen saturation level (Functional SpO2) for the user based on a ratio of the determined oxyhemoglobin blood content (V_{O2Hb}) to a sum of the determined the deoxyhemoglobin blood content (V_{HHb}) and the determined oxyhemoglobin blood content (V_{O2Hb}).

In embodiments, processing element 120 can select one or more optical transmitter arrays 128 of the optical transmitter and receiver assembly 106 for use with determining physiological characteristics for the user, such as a glycated hemoglobin (HbA1c) level, a heart rate, a heart rate variability, a blood pressure, peripheral oxygen saturation (e.g., SpO2), a stress intensity level, and a body energy level of the user based on a determined signal quality metric, such as signal-to-noise ratio, for the optical transmitter arrays 128. Processing element 120 may select an optical transmitter array 128 for output of optical signals to produce PPG signals having an acceptable cardiac component within the PPG signal.

The one or more optical transmitter arrays 128 may be selected by the processing element 120 based on an optimization of spectral properties of transmitted electromagnetic waves that would enable an optical receiver 110 to generate a PPG signal that may enable the processing element 120 to determine accurate physiological information for the user. Generally, the optical receiver 110 generates a PPG signal by converting an intensity of the optical signal (e.g., a visible or invisible electromagnetic wave) reflected from the user's skin after it has passed through human tissue from an optical transmitter 108 of an optical transmitter array 128. Typically, the intensity of reflected light measured (e.g., sensed) by the optical receiver 110 is modulated by the subject's cardiac cycle, which causes variation in tissue blood volume during the cardiac cycle as the user's heart beats. The intensity of measured light is also strongly influenced by many factors other than the cardiac cycle. The other factors may include ambient light intensity including static and variable, body motion at the measurement location, static and variable sensor pressure on the user's skin, motion of the optical transmitter and receiver assembly 106 relative to the body at the measurement location, motion of the user breathing, and light barriers (e.g., hair, opaque skin layers, sweat, etc.). Relative to these sources, the cardiac component of the PPG signal may be very weak. In some instances, the cardiac component of the PPG signal may be lower than the other factors by one or more orders of magnitude, which can result in the cardiac component of the PPG signal having a low signal quality metric, such as signal-to-noise ratio.

The memory element 122 can be a tangible, computer-readable storage medium that provides storage functionality to store various data and/or program code associated with an operation, such as software programs and/or code segments, or other data to instruct the processing element 120, and possibly other components of the wrist-worn electronic device 100, to perform the functionality described herein. The memory element 122 can store data, such as program instructions for operating the wrist-worn electronic device 100 including its components, and so forth. The memory element 122 can also store absorption constants for deoxyhemoglobin blood content (k_{A1}, k_{A2} and k_{A3}), absorption constants for oxyhemoglobin blood content (k_{B1}, k_{B2} and k_{B3}) and absorption constants for glycated hemoglobin blood content (k_{C1}, k_{C2} and k_{C3}), each absorption constant associated with one of a first wavelength, a second wavelength or a third wavelength, as well as signal path factors (c₁-c₃). In embodiments, the memory element 122 can store signal quality metric thresholds, such as a signal-to-noise ratio threshold, heart rate information and/or oxygenation information determined for the user.

It should be noted that while a single memory element 122 is described, a wide variety of types and combinations of memory (e.g., tangible, non-transitory memory) can be employed. The memory element 122 can be integral with the processing element 120, can comprise stand-alone memory, or can be a combination of both. Some examples of the memory element 122 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory elements, hard disk memory, external memory, and so forth. In a number of embodiments, the wrist-worn electronic device 100 and/or the memory element 122 can include removable integrated circuit card (ICC) memory, such as memory provided by a subscriber identity module (SIM) card, a universal subscriber identity module (USIM) card, a universal integrated circuit card (UICC), and so on.

The location determining element 130 generally determines a current geolocation of the wrist-worn electronic device 100 and may receive and process radio frequency (RF) signals from a multi-constellation global navigation satellite system (GNSS) such as the global positioning system (GPS) utilized in the United States, the Galileo system utilized in Europe, the GLONASS system utilized in Russia, or the like. The location determining element 130 may accompany or include an antenna to assist in receiving the satellite signals. The antenna may be a patch antenna, a linear antenna, or any other type of antenna that can be used with location or navigation devices. The location determining element 130 may include satellite navigation receivers, processing elements, controllers, other computing devices, or combinations thereof, and memory. The location determining element 130 may process a signal, referred to herein as a “location signal”, from one or more satellites that includes data from which geographic information such as the current geolocation is derived. The current geolocation may include coordinates, such as the latitude and longitude, of the current location of the wrist-worn electronic device 100. The location determining element 130 may communicate the current geolocation to the processing element 120, the memory element 122, or both.

Although embodiments of the location determining element 130 may include a satellite navigation receiver, it will be appreciated that other location-determining technology may be used. For example, cellular towers or any customized transmitting radio frequency towers can be used instead of satellites may be used to determine the location of the wrist-worn electronic device 100 by receiving data from at least three transmitting locations and then performing basic triangulation calculations to determine the relative position of the device with respect to the transmitting locations. With such a configuration, any standard geometric triangulation algorithm can be used to determine the location of the wrist-worn electronic device 100. The location determining element 130 may also include or be coupled with a pedometer, accelerometer, compass, or other dead-reckoning components which allow it to determine the location of the wrist-worn electronic device 100. The location determining element 130 may determine the current geographic location through a communications network, such as by using Assisted GPS (A-GPS), or from another electronic device, such as a fitness device or a mobile device (e.g., smartphone). The location determining element 130 may even receive location data directly from a user.

The display 132 generally presents the information mentioned above, such as time of day, current location, and the like. The display 132 may be implemented in one of the following technologies: light-emitting diode (LED), organic LED (OLED), Light Emitting Polymer (LEP) or Polymer LED (PLED), liquid crystal display (LCD), thin film transistor (TFT) LCD, LED side-lit or back-lit LCD, or the like, or combinations thereof. In some embodiments, the display 132 may have a round, circular, or oval shape. In other embodiments, the display 132 may possess a square or a rectangular aspect ratio which may be viewed in either a landscape or a portrait orientation.

The display 132 or user interface generally allows the user to directly interact with the wrist-worn electronic device 100 and may include pushbuttons, rotating knobs, or the like. In various embodiments, the display 132 may also include a touch screen occupying the entire display 132 or a portion thereof so that the display 132 functions as at least a portion of the user interface. The touch screen may allow the user to interact with the wrist-worn electronic device 100 by physically touching, swiping, or gesturing on areas of the display 132.

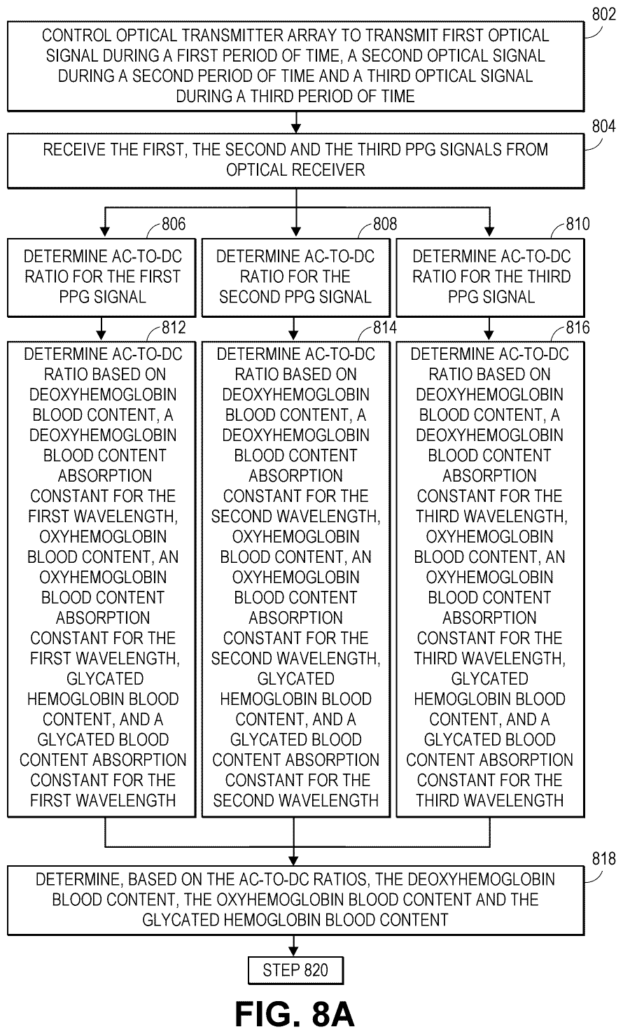

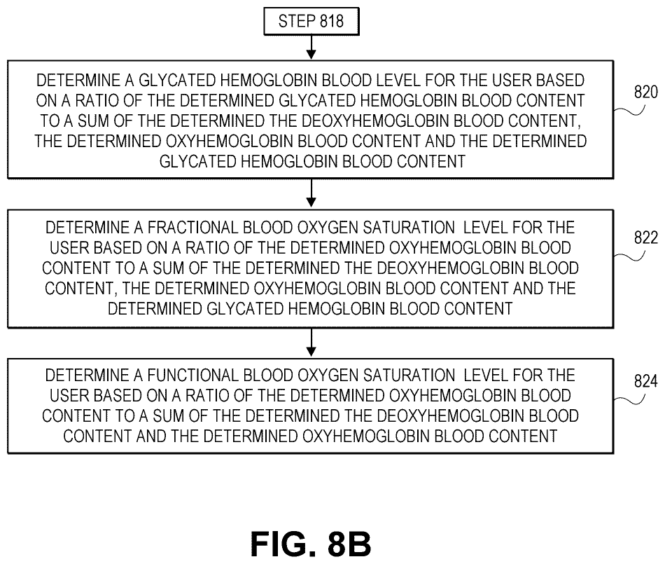

In embodiments, the processing element 120 may execute, process, or run instructions, code or software to perform the process steps shown in FIGS. 8A-8B. FIGS. 8A and 8B are a flowchart 800 for determining a glycated hemoglobin blood level (HbA1c), a fractional blood oxygen saturation level (Fractional SpO2) and/or a functional blood oxygen saturation level (Functional SpO2) in accordance with embodiments of the technology. In various embodiments, one or more regions of method 800 (or the entire method 800) may be implemented by any suitable device. Method 800 represents the calculations performed to calculate and display physiological information about the user using PPG signals output by the optical receiver 110 based on the reflections of a plurality of optical signals that are received from the user's skin. For example, method 800 may represent the iterative steps taken to generate PPG signals and calculate physiological information about the user, such as a glycated hemoglobin blood level (HbA1c), a fractional blood oxygen saturation level (Fractional SpO2) and/or a functional blood oxygen saturation level (Functional SpO2), as discussed herein, with the calculated physiological information being displayed via display 132.

Referring to process step 802, the processing element 120 is configured to control one or more optical transmitter array 128 to transmit first optical signal during a first period of time, a second optical signal during a second period of time and a third optical signal during a third period of time. Referring to process step 804, the processing element 120 receives the first, the second and the third PPG signals from one or more optical receivers 110.

Referring to process step 806, the processing element 120 determines a first AC-to-DC ratio for the first PPG signal. Referring to process step 808, the processing element 120 determines a second AC-to-DC ratio for the second PPG signal. Referring to process step 810, the processing element 120 determines a third AC-to-DC ratio for the third PPG signal.

Referring to process step 812, the processing element 120 determines a fourth AC-to-DC ratio associated with deoxyhemoglobin blood content (V_{HHb}), a first absorption constant for deoxyhemoglobin blood content corresponding to the first wavelength (k_{A1}), oxyhemoglobin blood content (V_{O2Hb}), a first absorption constant for oxyhemoglobin blood content corresponding to the first wavelength (k_{B1}), glycated hemoglobin blood content (V_{HbA1c}), and a first absorption constant for glycated blood content corresponding to the first wavelength (k_{C1}).

Referring to process step 814, the processing element 120 determines a fifth AC-to-DC ratio associated with deoxyhemoglobin blood content (V_{HHb}), a second absorption constant for deoxyhemoglobin blood content corresponding to the second wavelength (k_{A2}), oxyhemoglobin blood content (V_{O2Hb}), a second absorption constant for oxyhemoglobin blood content corresponding to the second wavelength (k_{B2}), glycated hemoglobin blood content (V_{HbA1c}), and a second absorption constant for glycated blood content corresponding to the second wavelength (k_{C2}).

Referring to process step 816, the processing element 120 determines a sixth AC-to-DC ratio associated with deoxyhemoglobin blood content (V_{HHb}), a third absorption constant for deoxyhemoglobin blood content corresponding to the third wavelength (k_{A3}), oxyhemoglobin blood content (V_{O2Hb}), a third absorption constant for oxyhemoglobin blood content corresponding to the third wavelength (k_{B3}), glycated hemoglobin blood content (V_{HbA1c}), and a third absorption constant for glycated blood content corresponding to the third wavelength (k_{C3}).

Referring to process step 818, the processing element 120 determines, based on the six determined AC-to-DC ratios, the deoxyhemoglobin blood content (V_{HHb}), the oxyhemoglobin blood content (V_{O2Hb}) and the glycated hemoglobin blood content (V_{HbA1c}).

Referring to process step 820, the processing element 120 determines a glycated hemoglobin blood level (HbA1c) for the user based on a ratio of the determined glycated hemoglobin blood content (V_{HbA1c}) to a sum of the determined the deoxyhemoglobin blood content (V_{HHb}), the determined oxyhemoglobin blood content (V_{O2Hb}) and the determined glycated hemoglobin blood content (V_{HbA1c}).

Referring to process step 822, the processing element 120 determines a fractional blood oxygen saturation level (Fractional SpO2) for the user based on a ratio of the determined oxyhemoglobin blood content (V_{O2Hb}) to a sum of the determined the deoxyhemoglobin blood content (V_{HHb}), the determined oxyhemoglobin blood content (V_{O2Hb}) and the determined glycated hemoglobin blood content (V_{HbA1c}).

Referring to process step 824, the processing element 120 determines a functional blood oxygen saturation level (Functional SpO2) for the user based on a ratio of the determined oxyhemoglobin blood content (V_{O2Hb}) to a sum of the determined the deoxyhemoglobin blood content (V_{HHb}) and the determined oxyhemoglobin blood content (V_{O2Hb}).

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

As used herein, “a number of” something can refer to one or more of such things. As will be appreciated, elements shown in the various embodiments herein can be added, exchanged, and/or eliminated so as to provide a number of additional embodiments of the present disclosure.

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either present technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

What is claimed is:

1. An electronic device, comprising:
a housing including a bottom wall configured to contact a user's skin;
an optical transmitter array positioned at a first location on the bottom wall and operable to output a plurality of optical signals that pass through a user's skin, the plurality of optical signals including a first optical signal having a first wavelength, a second optical signal having a second wavelength and a third optical signal having a third wavelength;
an optical receiver positioned at a second location on the bottom wall and operable to receive the optical signals modulated by the skin of the user and generate first, second and third photoplethysmogram (PPG) signals resulting from the received first, the second and the third optical signals, respectively, the first optical signal, the second optical signal and the third optical signal each traveling substantially along the same path and each optical signal containing a cardiac component of the user;
a memory element configured to store absorption constants for deoxyhemoglobin blood content, oxyhemoglobin blood content and glycated hemoglobin blood content, each absorption constant associated with one of the first wavelength, the second wavelength or the third wavelength; and
a processing element in electronic communication with the optical transmitter array and the optical receiver, the processing element configured to:
receive the first, the second and the third PPG signals from the optical receiver,
determine a first AC-to-DC ratio for the first PPG signal,
determine a second AC-to-DC ratio for the second PPG signal,
determine a third AC-to-DC ratio for the third PPG signal,
determine, based on the three determined AC-to-DC ratios, the deoxyhemoglobin blood content, the oxyhemoglobin blood content and the glycated hemoglobin blood content, and
determine a glycated hemoglobin blood level for the user based on a ratio of the determined glycated hemoglobin blood content to a sum of the determined the deoxyhemoglobin blood content, the determined oxyhemoglobin blood content and the determined glycated hemoglobin blood content.

2. The electronic device of claim 1, wherein the processing element is further configured to:
determine a fourth AC-to-DC ratio associated with deoxyhemoglobin blood content, a first absorption constant for deoxyhemoglobin blood content corresponding to the first wavelength, oxyhemoglobin blood content, a first absorption constant for oxyhemoglobin blood content corresponding to the first wavelength, glycated hemoglobin blood content, and a first absorption constant for glycated blood content corresponding to the first wavelength,
determine a fifth AC-to-DC ratio associated with deoxyhemoglobin blood content, a second absorption constant for deoxyhemoglobin blood content corresponding to the second wavelength, oxyhemoglobin blood content, a second absorption constant for oxyhemoglobin blood content corresponding to the second wavelength, glycated hemoglobin blood content, and a second absorption constant for glycated blood content corresponding to the second wavelength,
determine a sixth AC-to-DC ratio associated with deoxyhemoglobin blood content, a third absorption constant for deoxyhemoglobin blood content corresponding to the third wavelength, oxyhemoglobin blood content, a third absorption constant for oxyhemoglobin blood content corresponding to the third wavelength, glycated hemoglobin blood content, and a third absorption constant for glycated blood content corresponding to the third wavelength, and
determine, based on the six determined AC-to-DC ratios, the deoxyhemoglobin blood content, the oxyhemoglobin blood content and the glycated hemoglobin blood content.

3. The electronic device of claim 1, wherein the memory element is further configured to store a plurality of signal path factors, the plurality of signal path factors including a first signal path factor corresponding to the first wavelength, a second signal path factor corresponding to the second wavelength, and a third signal path factor corresponding to the third wavelength.

4. The electronic device of claim 3, wherein the processing element is further configured to determine the first AC-to-DC ratio for the first PPG signal based on the first signal path factor, determine the second AC-to-DC ratio for the second PPG signal based on the second signal path factor, and determine a third AC-to-DC ratio for the third PPG signal based on the third signal path factor.

5. The electronic device of claim 1, wherein the optical transmitter array includes a first optical transmitter configured to transmit the first optical signal, a second optical transmitter configured to transmit the second optical signal and a third optical transmitter configured to transmit the third optical signal, and wherein the processing element is further configured to control the first optical transmitter to transmit the first optical signal during a first period of time, the second optical transmitter to transmit the second optical signal during a second period of time and the third optical transmitter to transmit the third optical signal during a third period of time.

6. The electronic device of claim 1, wherein the processing element is further configured to control the optical transmitter array to transmit the first optical signal during a first period of time, the optical transmitter array to transmit the second optical signal during a second period of time and the optical transmitter array to transmit the third optical signal during a third period of time.

7. The electronic device of claim 6, wherein the first period of time, the second period of time and the third period of time are in sequential order.

8. The electronic device of claim 6, wherein the processing element is further configured to control the optical transmitter array to cause the first optical transmitter to transmit the first optical signal, the second optical signal and the third optical signal during a first period of time.

9. The electronic device of claim 6, wherein the optical transmitter array includes a first optical transmitter configured to transmit the first optical signal, the second optical signal and the third optical signal.

10. The electronic device of claim 6, wherein the optical transmitter array includes a first optical transmitter configured to transmit the first optical signal, a second optical transmitter configured to transmit the second optical signal and a third optical transmitter configured to transmit the third optical signal.

11. The electronic device of claim 1, wherein the first wavelength is between 620-650 nm and the second wavelength is between 650-700 nm.

12. The electronic device of claim 11, wherein the third wavelength is between one of 760 nm-860 nm and 900 nm-1,000 nm.

13. The electronic device of claim 1, further comprising a band, wherein the housing and band secure the electronic device to an extremity of the user.

14. An electronic device, comprising:
a housing including a bottom wall configured to contact a user's skin;
an optical transmitter array positioned at a first location on the bottom wall and operable to output a plurality of optical signals that pass through a user's skin, the plurality of optical signals including a first optical signal having a first wavelength, a second optical signal having a second wavelength and a third optical signal having a third wavelength;
an optical receiver positioned at a second location on the bottom wall and operable to receive the optical signals modulated by the skin of the user and generate first, second and third photoplethysmogram (PPG) signals resulting from the received first, the second and the third optical signals, respectively, the first optical signal, the second optical signal and the third optical signal each traveling substantially along the same path and each optical signal containing a cardiac component of the user;
a memory element configured to store absorption constants for deoxyhemoglobin blood content, oxyhemoglobin blood content and glycated hemoglobin blood content, each absorption constant associated with one of the first wavelength, the second wavelength or the third wavelength; and
a processing element in electronic communication with the optical transmitter array and the optical receiver, the processing element configured to:
receive the first, the second and the third PPG signals from the optical receiver,
determine a first AC-to-DC ratio for the first PPG signal,
determine a second AC-to-DC ratio for the second PPG signal,
determine a third AC-to-DC ratio for the third PPG signal,
determine a fourth AC-to-DC ratio associated with deoxyhemoglobin blood content, a first absorption constant for deoxyhemoglobin blood content corresponding to the first wavelength,
oxyhemoglobin blood content, a first absorption constant for oxyhemoglobin blood content corresponding to the first wavelength, glycated hemoglobin blood content, and a first absorption constant for glycated blood content corresponding to the first wavelength,
determine a fifth AC-to-DC ratio associated with deoxyhemoglobin blood content, a second absorption constant for deoxyhemoglobin blood content corresponding to the second wavelength, oxyhemoglobin blood content, a second absorption constant for oxyhemoglobin blood content corresponding to the second wavelength, glycated hemoglobin blood content, and a second absorption constant for glycated blood content corresponding to the second wavelength,
determine a sixth AC-to-DC ratio associated with deoxyhemoglobin blood content, a third absorption constant for deoxyhemoglobin blood content corresponding to the third wavelength, oxyhemoglobin blood content, a third absorption constant for oxyhemoglobin blood content corresponding to the third wavelength, glycated hemoglobin blood content, and a third absorption constant for glycated blood content corresponding to the third wavelength,
determine, based on the six determined AC-to-DC ratios, the deoxyhemoglobin blood content, the oxyhemoglobin blood content and the glycated hemoglobin blood content, and
determine a glycated hemoglobin blood level for the user based on a ratio of the determined glycated hemoglobin blood content to a sum of the determined the deoxyhemoglobin blood content, the determined oxyhemoglobin blood content and the determined glycated hemoglobin blood content.

15. The electronic device of claim 14, wherein the memory element is further configured to store a plurality of signal path factors, the plurality of signal path factors including a first signal path factor corresponding to the first wavelength, a second signal path factor corresponding to the second wavelength, and a third signal path factor corresponding to the third wavelength, and wherein the processing element is further configured to:
determine the first AC-to-DC ratio for the first PPG signal based on the first signal path factor,
determine the second AC-to-DC ratio for the second PPG signal based on the second signal path factor, and
determine a third AC-to-DC ratio for the third PPG signal based on the third signal path factor.

16. The electronic device of claim 14, further comprising a band, wherein the housing and band secure the electronic device to an extremity of the user, and wherein the first wavelength is between 620-650 nm and the second wavelength is between 650-700 nm.

17. An electronic device, comprising:
a housing including a bottom wall configured to contact a user's skin;
an optical transmitter array positioned at a first location on the bottom wall and operable to output a plurality of optical signals that pass through a user's skin, the plurality of optical signals including a first optical signal having a first wavelength, a second optical signal having a second wavelength and a third optical signal having a third wavelength;
an optical receiver positioned at a second location on the bottom wall and operable to receive the optical signals modulated by the skin of the user and generate first, second and third photoplethysmogram (PPG) signals resulting from the received first, the second and the third optical signals, respectively, the first optical signal, the second optical signal and the third optical signal each traveling substantially along the same path and each optical signal containing a cardiac component of the user;
a memory element configured to store:
absorption constants for deoxyhemoglobin blood content, oxyhemoglobin blood content and glycated hemoglobin blood content, each absorption constant associated with one of the first wavelength, the second wavelength or the third wavelength, and
a plurality of signal path factors, the plurality of signal path factors including a first signal path factor corresponding to the first wavelength, a second signal path factor corresponding to the second wavelength, and a third signal path factor corresponding to the third wavelength; and
a processing element in electronic communication with the optical transmitter array and the optical receiver, the processing element configured to:
receive the first, the second and the third PPG signals from the optical receiver,
determine a first AC-to-DC ratio for the first PPG signal based on the first signal path factor,
determine a second AC-to-DC ratio for the second PPG signal based on the second signal path factor,
determine a third AC-to-DC ratio for the third PPG signal based on the third signal path factor,
determine, based on the three determined AC-to-DC ratios, the deoxyhemoglobin blood content, the oxyhemoglobin blood content and the glycated hemoglobin blood content, and
determine a glycated hemoglobin blood level for the user based on a ratio of the determined glycated hemoglobin blood content to a sum of the determined the deoxyhemoglobin blood content, the determined oxyhemoglobin blood content and the determined glycated hemoglobin blood content.

18. The electronic device of claim 17, wherein the processing element is further configured to:
determine a fourth AC-to-DC ratio associated with deoxyhemoglobin blood content, a first absorption constant for deoxyhemoglobin blood content corresponding to the first wavelength, oxyhemoglobin blood content, a first absorption constant for oxyhemoglobin blood content corresponding to the first wavelength, glycated hemoglobin blood content, and a first absorption constant for glycated blood content corresponding to the first wavelength,
determine a fifth AC-to-DC ratio associated with deoxyhemoglobin blood content, a second absorption constant for deoxyhemoglobin blood content corresponding to the second wavelength, oxyhemoglobin blood content, a second absorption constant for oxyhemoglobin blood content corresponding to the second wavelength, glycated hemoglobin blood content, and a second absorption constant for glycated blood content corresponding to the second wavelength,
determine a sixth AC-to-DC ratio associated with deoxyhemoglobin blood content, a third absorption constant for deoxyhemoglobin blood content corresponding to the third wavelength, oxyhemoglobin blood content, a third absorption constant for oxyhemoglobin blood content corresponding to the third wavelength, glycated hemoglobin blood content, and a third absorption constant for glycated blood content corresponding to the third wavelength, and
determine, based on the six determined AC-to-DC ratios, the deoxyhemoglobin blood content, the oxyhemoglobin blood content and the glycated hemoglobin blood content.

19. The electronic device of claim 17, wherein the processing element is further configured to determine the first signal path factor, the second signal path factor and the third signal path factor to account for differences in the substantially the same signal paths between the optical transmitter array and the optical receiver, the differences including a region of the user's skin each of the first optical signal, the second optical signal and the third optical signal pass based on the first wavelength, the second wavelength or the third wavelength, respectively.

20. The electronic device of claim 18, wherein the first wavelength is between 620-650 nm, the second wavelength is between 650-700 nm and the third wavelength is between 900 nm-1,000 nm.
